SEQUENCE LISTING (2 sequences)

SEQ ID NO: 1 - protein, 164 aa, Homo sapiens
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 2 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

(19) United States
(12) Patent Application Publication
(10) Pub. No.: US 2019/0184015 A1
(43) Pub. Date: Jun. 20, 2019

(54) IRRADIATION TREATMENT OF NEUROLOGICAL SENSATIONS BY PHOTOABLATION

(71) Applicant: European Molecular Biology Laboratory, Heidelberg (DE)

(72) Inventors: Paul Heppenstall, Monterotondo (IT); Rahul Dhandapani, Chennai (IN); Carla Portulano, Taranto (IT)

(21) Appl. No.: 16/329,904

(22) PCT Filed: Sep. 1, 2017
(86) PCT No.: PCT/EP2017/072014

§ 371 Date: Mar. 1, 2019

(30) Foreign Application Priority Data
Sep. 2, 2016 (EP) 16187087.8
Jun. 27, 2017 (EP) 17178123.0

(51) Int. Cl. A61K 41/00; A61K 31/695; A61K 38/20; A61K 47/54; A61K 38/16; A61P 17/04; A61P 25/02; A61K 38/18

(52) U.S. Cl. CPC A61K 41/0071; A61K 31/695; A61K 38/20; A61K 38/185; A61K 38/164; A61P 17/04; A61P 25/02; A61K 47/558

(57) ABSTRACT

The present invention relates to an approach for the treatment of adverse neurological sensations in a certain body surface area such as the skin, in particular treatment of pain or itching. The invention is based on the finding that administration of a targeting molecule which specifically binds a cell or receptor responsible for the adverse sensation in the respective body surface area of a patient, and which is coupled/conjugated to a photosensitive inhibitor or cytotoxic agent can enable the irradiation dependent ablation of cells responsible for the sensation. This approach allows a targeted and specific treatment of body surface areas by irradiation. Provided are conjugate compounds for use in the photoablation treatment of the invention and pharmaceutical compositions which comprise these compounds.

The present invention relates to an approach for the treatment of adverse neurological sensations in a certain body surface area such as the skin, in particular treatment of pain or itching. The invention is based on the finding that administration of a targeting molecule which specifically binds a cell or receptor responsible for the adverse sensation in the respective body surface area of a patient, and which is coupled/conjugated to a label, photosensitive inhibitor or cytotoxic agent can enable the irradiation dependent ablation and/or retraction of cells responsible for the sensation. This approach allows a targeted and specific treatment of body surface areas by irradiation. Provided are conjugate compounds for use in the photoablation treatment of the invention and pharmaceutical compositions which comprise these compounds.

### BACKGROUND OF THE INVENTION

Itch is a cutaneous sensory perception defined by the behavioral response it elicits: an urgent need to scratch (A. Ikoma, M. Steinhoff, S. Stander, G. Yosipovitch, M. Schmelz, The neurobiology of itch. Nat Rev Neurosci 7, 535-547 (2006)). When itching becomes pathological, it can be irritating and distressful and have a dramatic impact on quality of life (S. Davidson, G. J. Giesler, The multiple pathways for itch and their interactions with pain. Trends Neurosci 33, 550-558 (2010)). Chronic itch generates a recurrent cycle whereby the more the skin is scratched, the more it itches (C. F. Wahlgren, Itch and atopic dermatitis: an overview. J Dermatol 26, 770-779 (1999)). In turn, this may lead to serious damage to the skin barrier and thereby an increased risk of infection. There are many itch-associated diseases such as atopic dermatitis, eczema and psoriasis that respond poorly to current therapies (S. B. Elmariah, E. A. Lerner, Topical therapies for pruritus. Semin Cutan Med Surg 30, 118-126 (2011)). Identifying novel strategies to reduce itching is therefore critical and requires a deeper understanding of the underlying mechanisms.

Although several key molecules involved in transducing itch sensation have recently been described, the cellular and molecular mechanisms that drive chronic itch are not fully understood. The transduction of itch begins in the skin where a network of different cell types, such as keratinocytes, sensory nerves and immune cells respond to exogenous or endogenous pruritogens and initiate the cascade which ends with the scratch response (D. M. Bautista, S. R. Wilson, M. A. Hoon, Why we scratch an itch: the molecules, cells and circuits of itch. Nat Neurosci 17, 175-182 (2014)). Two major itch pathways have been described; the histaminergic pathway which is mediated by histamine (K. Rossbach et al., Histamine H1, H3 and H4 receptors are involved in pruritus. Neuroscience 190, 89-102 (2011)), and the non-histaminergic pathway which includes other itch mediators, such as chloroquine and inflammatory cytokines (Q. Liu et al., Sensory neuron-specific GPCR Mrgprs are itch receptors mediating chloroquine-induced pruritus. Cell 139, 1353-1365 (2009)). It is becoming increasingly apparent that anti-histaminergic drugs are often ineffective against many chronic itch conditions such as atopic dermatitis and psoriasis (A. Reich, J. C. Szepietowski, Mediators of pruritus in psoriasis. Mediators Inflamm 2007, 64727 (2007), and N. Takano, I. Arai, Y. Hashimoto, M. Kurachi, Evaluation of antipruritic effects of several agents on scratching behavior by NC/Nga mice. Eur J Pharmacol 495, 159-165 (2004)). Attention has now turned to non-histaminergic pathways to control itch sensation under pathological conditions (M. Steinhoff et al., Neurophysiological, neuroimmunological, and neuroendocrine basis of pruritus. J Invest Dermatol 126, 1705-1718 (2006)).

Amongst anti-histamine resistant itch mediators, the cytokine Interleukin 31 (IL-31) has recently attracted much attention as a novel target molecule for chronic itch therapy (S. R. Dillon et al., Interleukin 31, a cytokine produced by activated T cells, induces dermatitis in mice. Nat Immunol 5, 752-760 (2004)). IL-31 is a four-helix bundle cytokine with a prominent skin tropism that is produced preferentially by T helper-type 2 cells (O. Grimstad et al., Anti-interleukin-31-antibodies ameliorate scratching behaviour in NC/Nga mice: a model of atopic dermatitis. Exp Dermatol 18, 35-43 (2009)). It signals through a heterodimeric receptor composed of IL31RA and OSMR which are expressed in epithelial cells, keratinocytes and sensory neurons (Bando, Y. Morikawa, T. Komori, E. Senba, Complete overlap of interleukin-31 receptor A and oncostatin M receptor beta in the adult dorsal root ganglia with distinct developmental expression patterns. Neuroscience 142, 1263-1271 (2006), and C. Diveu et al., Predominant expression of the long isoform of GP130-like (GPL) receptor is required for interleukin-31 signaling. Eur Cytokine Netw 15, 291-302 (2004)). Transgenic mice overexpressing IL-31 develop severe pruritus, alopecia and skin lesions that resemble lesioned skin from patients with atopic dermatitis. Moreover, numerous studies have reported an association of IL-31 with inflammatory skin diseases with a severe pruritic component. For example, IL-31 mRNA is up-regulated in human patients with atopic dermatitis (E. Sonkoly et al., IL-31: a new link between T cells and pruritus in atopic skin inflammation. J Allergy Clin Immunol 117, 411-417 (2006)) and in mouse models of this disease (A. Takaoka et al., Expression of IL-31 gene transcripts in NC/Nga mice with atopic dermatitis. Eur J Pharmacol 516, 180-181 (2005)). Furthermore, a common IL31 haplotype has been associated with non-atopic eczema in three independent European populations, marking this as the first genetic risk factor for non-atopic eczema (F. Schulz et al., A common haplotype of the IL-31 gene influencing gene expression is associated with nonatopic eczema. J Allergy Clin Immunol 120, 1097-1102 (2007)). Intriguingly, it has been suggested that the major pathology evoked by IL-31 is to induce pruritus, rather than directly causing skin lesions per se (Q. Zhang, P. Putheti, Q. Zhou, Q. Liu, W. Gao, Structures and biological functions of IL-31 and IL-31 receptors. Cytokine Growth Factor Rev 19, 347-356 (2008)). Thus therapies which reduce scratching and break the cycle of itch and disruption of the skin's barrier function (M. W. Greaves, N. Khalifa, Itch: more than skin deep. International archives of allergy and immunology 135, 166-172 (2004)) may be the most effective strategies for improving the quality of life for patients with chronic pruritic disease.

Another neurological sensation covered by the invention is pain. The treatment of pain conditions is of great importance in medicine. There is currently a world-wide need for additional pain therapy. The pressing requirement for a specific treatment of pain conditions or as well a treatment of specific pain conditions which is right for the patient, which is to be understood as the successful and satisfactory treatment of pain for the patients, is documented in the large number of scientific works which have recently and over the years appeared in the field of applied analgesics or on basic research on nociception. PAIN is defined by the International Association for the Study of Pain (IASP) as “an unpleasant sensory and emotional experience associated with actual or potential tissue damage, or described in terms of such damage” (IASP, Classification of chronic pain, 2nd Edition, IASP Press (2002), 210). Even though pain is always subjective its causes or syndromes can be classified.

In neuropathic pain patients, hypersensitivity to light touch can develop to the extent that movement of a single hair shaft is sufficient to provoke severe pain. This impacts greatly upon quality of life due to the pervasive nature of mechanical stimuli (mechanical allodynia); for example, small movements of the body, or the weight of clothing can cause severe pain in neuropathic patients. While much recent progress has been made in delineating the spinal circuits that gate mechanical pain, however the sensory neurons that input this sensation into the spinal cord are not known.

Especially mechanical allodynia which in the past years has developed into a major health problem in broad areas of the population needs a very specific treatment, especially considering that any treatment of mechanical allodynia is extremely sensitive to the causes behind the pain, be it the disease ultimately causing it or the mechanistic pathway over which it develops.

Hypothetically, mechanical hypersensitivity could be mediated either by sensitization of nociceptors, or through integration of input from low threshold mechanoreceptors into pain transmitting circuits. In human studies, there is little evidence for nociceptor sensitization, and most reports indicate that mechanical allodynia is conveyed by myelinated A-fibre mechanoreceptors. For example, differential block of these nerves alleviates brush evoked pain, and the short latency of pain perception is indicative of the fast conduction velocity of A-fibres. Similarly, in experimental animal studies it has been demonstrated that mice develop mechanical allodynia in neuropathic pain models even when all nociceptors are genetically ablated. Unmyelinated C-low threshold mechanoreceptors marked by Vglut3 expression were initially proposed as a candidate population for driving mechanical hypersensitivity. However, allodynia persists even when these neurons fail to develop, and recent evidence indicates that transient Vglut3 expression in spinal interneurons accounts for the phenotype. Thus a subtype of A-fibre mechanoreceptor is likely the input that drives pain sensation from innocuous touch stimulation.

Therefore, it was the underlying problem of this invention to develop a therapeutic strategy for the treatment of adverse neurological sensations in body surface areas such as parts of the skin which overcomes the aforementioned drawbacks exemplified for the treatment of itch and pain.

In a first aspect of the invention, the above problem is solved by a method for targeting and inhibiting/killing a target cell in a target body surface area of a subject, the method comprising the steps of:
(a) Providing a conjugate compound, preferably a protein conjugate compound, comprising (i) a binding domain which specifically binds to the target cell, and (ii) a photosensitive inhibition/cytotoxin group,
(b) Administering said conjugate compound to the subject, and
(c) Irradiating said target body surface area of the subject with an appropriate excitation light in an amount to effectively activate said photosensitive inhibition/cytotoxin group and to induce cellular inhibition or cell death of the target cell.

In some embodiments it is preferred that the administration of the conjugate compound to the subject results in a sufficient final concentration of the conjugate compound in the respective target body surface area of the subject.

In certain aspects the invention pertains to the above noted conjugate compound for use in such a method, or alternatively for use in the treatment of neurological sensations in a body surface area. Preferably, the neurological sensation is selected from noxious or innocuous stimuli, such as all forms of mechanical (touch) sensation, pain and/or itching. Preferred is in some embodiments that the neurological sensation is a sensation other than pain, such as itch.

Preferably, in some embodiments, the method for targeting and inhibiting/killing a target cell in a target body surface area of a subject is a method for treating a neurological sensation in said subject. In this case the target cell is a cell mediating, or being involved in, the pathology or manifestation of said neurological sensation. Further, the target body surface area is an area in which the subject perceives, feels, experiences or otherwise senses (locally) said neurological sensation, or parts of it. In some embodiments of the invention the target cell is a (for example peripheral) sensory neuron(s), preferably expressing Tropomyosin receptor kinase B (TrkB), Tropomyosin receptor kinase A (TrkA) or alternatively IL31RA and/or Oncostatin-M specific receptor (OSMR).

The terms “neurological sensation” and “sensation” are used interchangingly and shall refer to any, preferably discomforting, neurological experience of a subject that can be localized to a discrete body surface area. Preferred examples of neurological sensations in context of the invention are touch sensations and/or sensations of itch or pain.

The term “itch” is herein used interchangeably with the term pruritus and intended to have the same meaning. It is a condition characterized by an unpleasant skin sensation, leading to the desire to scratch the respective area. “Itch” or “pruritus” can be a symptom of many diseases, disease states, or disorders. It may also be present independently of a disease, disease state, or disorder. The term “itch” or “pruritus” includes itch, or pruritus, wherein the cause of the itch or pruritus is associated with or due to a disorder, disease or disease state, and includes itch or pruritus wherein the cause or origin is not understood. “Itch related disorder or disease” is known in the field. The term “itch related disorder or disease” means itch associated with or due to a disorder or disease. Accordingly, “itch related disorder or disease” means “pruritus related disorder or disease”, which means “pruritus associated with or due to a disorder or disease”. “Disorder or disease” includes dermatological disease, systemic disease and neurological disorders with respect to the aforementioned sensations.

The patient to be treated using the invention described herein is preferably a human. In an alternative embodiment, the invention provides the treatment of a non-human mammal, preferably a dog or cat,

“Itch” or an “itch related disorder or disease”, particularly includes pruritoceptive itch, neurogenic itch, neuropathic itch, psychogenic itch and itch behaviors. More specifically, this includes pruritoceptive itch (originating in the skin, including itching arising from or associated with inflammatory skin diseases, e.g. skin diseases responsive to corticosteroid treatment and/or calcineurin inhibitor treatment, e.g. pimecrolimus, tacrolimus, cyclosporin A), neuropathic itch (due to a primary neurological disorder), neurogenic itch (arising from neurophysiological dysfunction) and idiopathic itch (itch of unknown cause e.g. idiopathic itch of the elderly (“senile pruritus” or chronic scalp itch).

Other embodiments of the invention pertain to all kinds of pain as neurological sensation. Within the context of the present invention, the term “pain” as used herein refers to a pain state experienced by a human individual or a mammal (also referred to as a “subject!” or “patient” herein) that includes a non-nociceptive pain, i.e., a neuropathic pain, a sympathetic pain, or both. As used herein, the term “pain” is also intended to include a mixed pain syndrome that includes a nociceptive pain state in addition to a non-nociceptive pain state. As used herein, the term “neuropathic pain” is a common type of chronic, non-malignant pain, which is the result of an injury or malfunction in the peripheral or central nervous system and serves no protective biological function. It may occur, for example, due to trauma, surgery, herniation of an intervertebral disk, spinal cord injury, diabetes, infection with herpes zoster, HIV/AIDS, late-stage cancer, amputation (including mastectomy), carpal tunnel syndrome, chronic alcohol use, exposure to radiation, and as an unintended side-effect of neurotoxic treatment agents, such as certain anti-HIV and chemotherapeutic drugs. In contrast to nociceptive pain, neuropathic pain is frequently described as “burning,” “electric,” “tingling,” or “shooting” in nature. It is often characterized by allodynia defined as pain resulting from a stimulus that does not ordinarily elicit a painful response such as light touch, and hyperalgesia defined as an increased sensitivity to a normally painful stimulus, and may persist for months or years beyond the apparent healing of any damaged tissues. One form of pain according to the invention is “mechanical allodynia”, which refers to the abnormal perception of pain from usually light mechanical stimulation, among allodynia which occurs due to a non-noxious stimulus that does not normally provoke pain, and it is the most severe neuropathic pain.

The present invention, its methods and compounds/compositions, pertain to the treatment of mechanical allodynia among traumatic or injurious pain such as postsurgical pain; metabolic pain such as diabetic neuropathy; ischemic or hemorrhagic pain such as central pain after stroke; toxic pain such as heavy metal poisoning or chemotherapy; compression pain such as spinal stenosis or carpal tunnel syndrome; immune-mediated pain such as multiple sclerosis; inflammatory pain such as post-herpetic neuralgia and hereditary pain such as Fabry's disease. The invention can also be used for the treatment of mechanical allodynia in the orofacial area.

Some embodiments of the application pertain in particular to the alleviation of nociceptive and inflammatory pain in a subject. Preferably the compounds and methods relating to the TrkA receptor and NGF fall in this category, and preferably shall be used in context of inflammatory pain. The term “nociceptive pain” refers to acute pain that arises under normal basal conditions, for example that associated with noxious mechanical, thermal or chemical stimuli. The term “inflammatory pain” or a pain associated with inflammation is intended to describe the subset of acute and chronic pain that results from inflammatory processes, such as may arise in the case of arthritis, infections and neoplasia or tumor related hypertrophy. Inflammatory pain includes pain associated with osteo-arthritis, rheumatoid arthritis, psoriatic arthropathy, arthritis associated with other inflammatory and autoimmune conditions, degenerative conditions such as back strain and mechanical back pain or disc disease, post operative pain, pain from an injury such as a soft tissue bruise or strained ligament or broken bone, abscess or cellulitis, fibrositis or myositis, Felty's syndrome, Sjogren's syndrome, peripheral neuropathy, biorythmus, bunions, burstis of the knee, Celiac's disease, Cushing syndrome, Costochondritis and Teize's syndrome, dry eyes, ganglion, juvenile idiopathic arthritis (juvenile rheumatoid arthritis), scleritis, relapsing polychondritis, pleurisy, connective tissue disease, steroid drug withdrawal, amyloidosis, uveitis, Raynard's phenomenon, osteopenia, chronic pain, Still's disease, swollen lymph nodes, Lyme disease, gout, sacroliac joint dysfunction, knee pain, lupus and ankle pain. In an embodiment, the pain is neuropathic pain such as but not limited to neuropathic and nociceptive aspects of osteo-arthritic pain.

Other examples of inflammatory conditions associated with pain include, but are not limited to, inflammatory diseases and disorders which result in a response of redness, swelling, pain, and a feeling of heat in certain areas that is meant to protect tissues affected by injury or disease. Inflammatory diseases which include a pain component which can be relieved using the compositions and methods of the present invention include, without being limited to, acne, angina, arthritis, aspiration pneumonia, disease, empyema, gastroenteritis, inflammation, intestinal flu, NEC, necrotizing enterocolitis, pelvic inflammatory disease (PID), pharyngitis, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy, chronic inflammatory demyelinating polyradiculoneuropathy.

Tropomyosin receptor kinase B (TrkB), also known as tyrosine receptor kinase B, or BDNF/NT-4 growth factors receptor or neurotrophic tyrosine kinase, receptor, type 2 is a protein that in humans is encoded by the NTRK2 gene. TrkB is a receptor for brain-derived neurotrophic factor (BDNF). TrkB is the high affinity catalytic receptor for several “neurotrophins”, which are small protein growth factors that induce the survival and differentiation of distinct cell populations. The neurotrophins that activate TrkB are: BDNF (Brain Derived Neurotrophic Factor), neurotrophin-4 (NT-4), and to a lesser extent neurotrophin-3 (NT-3). As such, TrkB mediates the multiple effects of these neurotrophic factors, which includes neuronal differentiation and survival. The TrkB receptor is part of the large family of receptor tyrosine kinases.

Tropomyosin receptor kinase A (TrkA), also known as high affinity nerve growth factor receptor, neurotrophic tyrosine kinase receptor type 1, or TRK1-transforming tyrosine kinase protein is a protein that in humans is encoded by the NTRK1 gene. This gene encodes a member of the neurotrophic tyrosine kinase receptor (NTKR) family. This kinase is a membrane-bound receptor that, upon ligand, such as nerve growth factor (NGF), binding, phosphorylates itself (autophosphorylation) and members of the MAPK. NGF mediated TrkA signaling is associated with pain, and in particular inflammatory pain.

In some embodiments of the invention it is preferred that the target cell is a neuron, preferably a sensory neuron. The term “sensory neuron” shall in preferred embodiments pertain to peripheral sensory neurons. As used herein, the term “peripheral sensory neuron” refers to a neuron located in the peripheral nerve system that receives and transmits information relating to sensory input, e.g. stimuli such as heat, touch, pressure, cold, vibration, itch etc. Preferred are mechanoreceptors. Preferably the one or more peripheral sensory neuron(s) is a myelinated neuron that innervates hair follicles. Sensory neurons that mediate itch are well known in the art (Lamotte R H et al: “Sensory neurons and circuits mediating itch”, NATURE REVIEWS NEUROSCIENCE, vol. 15, no. 1, 20 Dec. 2013 (2013 Dec. 20), pages 19-31, XP055152428, ISSN: 1471-003X, DOI: 10.1038/nrn3641).

In certain embodiments the conjugate compound is a molecule comprising a protein chain. It is particularly preferred that binding domain of the conjugate compound of the invention is provided by a protein, protein fragment or proteinaceous molecule. Such binding domains could be full length, or binding fragments of, protein ligands that are known to bind a receptor specifically expressed on a target cell of the invention, antibody molecules that bind to receptors or other cellular structures expressed on or in a target cell of the invention, or any other small- or macromolecular structure that allow for a specific targeting and binding of a target cell in accordance with the invention. Thus preferred is that the binding domain specifically binds to a receptor expressed on the cell. More preferred is that the binding domain is a receptor ligand, or a receptor binding fragment thereof, or a receptor binding antibody, or a receptor binding fragment thereof.

The term “antibody” or “antibodies” as used herein refers to monoclonal or polyclonal antibodies. The term “antibody” or “antibodies” as used herein includes but is not limited to recombinant antibodies that are generated by recombinant technologies as known in the art. Included are antibodies' of any species, in particular of mammalian species, including antibodies having two essentially complete heavy and two essentially complete light chains, human antibodies of any isotype, including IgAi, lgA2, IgD, Igd, lgG2a, lgG2b, lgG3, lgG IgE and IgM and modified variants thereof, non-human primate antibodies, e.g. from chimpanzee, baboon, rhesus or cynomolgus monkey, rodent antibodies, e.g. from mouse, rat or rabbit; goat or horse antibodies, and camelid antibodies (e.g. from camels or llamas such as Nanobodies™) and derivatives thereof, or of bird species such as chicken antibodies or of fish species such as shark antibodies. The term “antibody” or “antibodies” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody performance. Humanization reduces the immunogenicity of non-human antibodies in humans, thus facilitating the application of antibodies to the treatment of human disease. Humanized antibodies and several different technologies to generate them are well known in the art.

The term “antibody” or “antibodies” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies with specificity against a particular antigen upon immunization of the transgenic animal carrying the human germ-line immunoglobulin genes with said antigen. Technologies for producing such transgenic animals and technologies for isolating and producing the human antibodies from such transgenic animals are known in the art. Alternatively, in the transgenic animal; e.g. mouse, only the immunoglobulin genes coding for the variable regions of the mouse antibody are replaced with corresponding human variable immunoglobulin gene sequences. The mouse germline immunoglobulin genes coding for the antibody constant regions remain unchanged. In this way, the antibody effector functions in the immune system of the transgenic mouse and consequently the B cell development are essentially unchanged, which may lead to an improved antibody response upon antigenic challenge in vivo. Once the genes coding for a particular antibody of interest have been isolated from such transgenic animals the genes coding for the constant regions can be replaced with human constant region genes in order to obtain a fully human antibody. Other methods for obtaining human antibodies antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” or “antibodies” as used herein, also refers to an aglycosylated antibody. The term “antibody” or “antibodies” as used herein not only refers to untruncated antibodies of any species, including from human (e.g. IgG) and other mammalian species, but also refers to an antibody fragment. A fragment of an antibody comprises at least one heavy or light chain immunoglobulin domain as known in the art and binds to one or more antigen(s). Examples of antibody fragments according to the invention include Fab, Fab′, F(ab′)2, and Fv and scFv fragments; as well as diabodies, triabodies, tetrabodies, minibodies, domain antibodies(dAbs), such as sdAbs, VHH and VNAR fragments, single-chain antibodies, bispecific, trispecific, tetraspecific or multispecific antibodies formed from antibody fragments or antibodies, including but not limited to Fab-Fv or Fab-Fv-Fv constructs. Antibody fragments as defined above are known in the art. All antibodies as used in context of the invention are specifically and/or selectively binding to a target cell according to the invention, for example by binding to a specific molecular structure such as a receptor specifically expressed on said target cell. In some embodiments relating to the pain aspect of the invention the antibody according to the invention may specifically bind TrkB, TrkA or any other known pain specific receptor. With regard to the embodiments pertaining to itching or similar sensations, it is in some embodiments preferred that the antibody specifically binds to IL31 receptor (IL31RA) or any other known itch specific or itch mediating receptor.

In preferred embodiments the proteins and genes mentioned in this application are of mammalian origin, preferably of human origin.

A receptor which is a target for the binding domain of the conjugate compound of the invention is in preferred embodiments is specifically expressed in the target cell or target cell type. Such a receptor is preferably specifically involved or associated with the neurological sensation mediated by the target cell.

In context of the herein disclosed invention the cellular inhibition or cell death is neuronal, preferably axonal, retraction and/or inactivation. The term “inactivating” as used in context of the invention shall refer to a process of impairing the function of the sensory neuron as a neuronal transmitter of signals, for example signals caused by pathological malfunction of the cell or caused by, for example mechanical, stimuli. The invention may comprise as an inactivation any process that will reduce or inhibit the electrical propagation of a signal induced via a sensory neuron expressing the receptor or target of the invention, or impairing synaptic transmission of such neurons. In some embodiments inactivating one or more target cells, comprises inducing cytotoxicity in one or more target cells.

The conjugate compound in accordance with the herein disclosed invention comprises a photosensitive inhibition/cytotoxin group. In context of the invention such a inhibition/cytotoxin group may be selected from a functional group that acts as a cytotoxic agent. Examples for suitable effector groups are radioisotopes or radionuclides (e.g., ³H, ¹⁴O, ³⁵S, ⁹⁰Y, ⁹⁹Tc, ¹¹¹In, ¹²⁵I, ¹³¹I). Other suitable groups include toxins, therapeutic groups, or chemotherapeutic groups. Examples of suitable groups include calicheamicin, auristatins, geldanamycin and maytansine. In some embodiments, the effector group is coupled to the antigen binding protein via spacer arms of various lengths to reduce potential steric hindrance.

A preferred photosensitive inhibition/cytotoxin group of the invention is a photosensitizing agent, such as phthalocyanine IRDye®700DX or a derivative thereof, such as benzylguanine modified phthalocyanine IRDye®700DX. However, in other embodiments the photosensitizing agent is selected from a benzoporphyrin monoacid ring A (BPD-MA), tin etiopurpurin (SnET2), sulfonated aluminum phthalocyanine (AISPc) and lutetium texaphyrin (Lutex).

It is a beneficial effect of the method of the invention that only a subset of target cells responsible for a specific neurological sensation such as pain or itch are targeted by the conjugate compound. Therefore in a preferred embodiment of the invention pertains to the methods and compounds of the invention for use in a specific treatment, wherein the treatment essentially exclusively alleviates the neurological sensation mediated by the target cell, and no other forms of neuronal perception in a subject. Hence, the compounds and methods of the invention provide treatment options with reduced adverse or other side effects compared to prior art methods which often un-specifically impair multiple neurological sensations. Thus, the method of the invention in some embodiments is for alleviating a neurological sensation in the target body surface area of the subject.

In preferred embodiments of the invention the conjugate compound comprises a pruritogen as a binding domain which specifically binds to the cell. The term “pruritogen” as used in context of the invention shall refer to any compounds inducing an itching sensation in a subject. Preferably a pruritogen in context of the invention is a molecule binding to a receptor involved in or associated with the neurological circuit mediating itching sensation. More preferably, the pruritogen according to the invention is interleukin-31 (IL31) or mutant IL31, or derivatives or fragments of these compounds.

Therefore, in preferred embodiments of the invention the conjugate compound comprises a IL31, or mutant IL31, conjugated to phthalocyanine dye IRDye® 700DX, or a derivative hereof, such as a benzylguanine modified derivative.

A mutant IL-31 in accordance with the invention is preferably an IL31 binding to IL31 receptor (Il31RA and OSMR), but eliciting a reduced IL31 signaling, such as is IL31^{K134A}. IL31 is preferably a protein having an amino acid sequence as shown in SEQ ID NO:1, or a variant thereof having at least 70%, 75%, 80%, 85%, 90%, 95%, 96%, 97%, 98%, preferably 99% sequence identity to the amino acid sequence shown in SEQ ID NO: 1.

In some preferred embodiments of the invention the conjugate compound comprises a Il31, or mutant IL31, conjugated via a SNAP tag to phthalocyanine dye IRDye® 700DX, or a derivative hereof, such as a benzylguanine modified derivative. Also more generally, the use of a SNAP tag or other equivalent tag systems are preferred to conjugate the binding domain (such as IL31 or mutant IL31, or TrkA−, or TrkB ligands) with the photosensitive cytotoxin/inhibition group.

In one alternative embodiment of the invention the binding domain which specifically binds to the target cell is capable to bind to an expression product of a TrkB gene, preferably the NTRK2 gene, in the target cell. This embodiment is useful in the context of pain. The expression product of the TrkB gene is a TrkB protein or a TrkB RNA, preferably TrkB mRNA. For example, the compound that is capable to bind to an expression product of the TrkB gene comprises a TrkB-ligand or an anti-TrkB-antibody or anti-TrkB-T cell receptor (TCR), or chimeric antigen receptor (CAR); or wherein the compound comprises a nucleic acid having a nucleic acid sequence that is complementary to, or can under stringent conditions hybridize to, an mRNA produced by the NTRK2 locus. Specific examples include a protein binding to the TrkB/p75 receptor complex, and preferably is selected from Brain-derived neurotrophic factor (BDNF) or Neurotrophin 4 (NT-4).

In another aspect there is provided a conjugate compound that is capable of binding to TrkB, comprising a binding fragment of a TrkB ligand or a TrkB ligand, and which is conjugated to a cytotoxic agent and/or label. The conjugate compound according to this aspect is preferred in some embodiments, when the binding fragment of a TrkB ligand comprises the amino acid sequence of Brain-derived neurotrophic factor (BDNF) or Neurotrophin 4 (NT-4), and wherein the label or cytotoxic agent is a photosensitizing agent.

For the herein disclosed embodiments and aspects of the invention the photosensitive inhibition/cytotoxin group may in some embodiments be a label or labeling group. The term “label” or “labeling group” refers to any detectable label. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labeling group is coupled to the antigen binding protein following: radioisotopes or radionuclides (e.g., 3H, 14O, 35S, 90Y, 99Tc, 111In, 125I, 131I) fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the TrkB binding compound via spacer arms of various lengths to reduce potential steric hindrance. Various methods for labeling compounds/proteins are known in the art and may be used as is seen fi

Preferably the binding fragment of a TrkB ligand comprises the amino acid sequence of Brain-derived neurotrophic factor (BDNF) or Neurotrophin 4 (NT-4), or an amino acid sequence that is at least 70%, preferably 80%, 90%, or most preferably 95% identical to the amino acid sequence of BDNF or Nt-4, preferably human BDNF or Nt-4; and wherein the label or cytotoxic agent is a photosensitizing agent. The amino acid sequences of human BDNF and NT-4 are preferably the amino acid sequences of a protein expressed by the BDNF or NT-4 genes respectively. The BDNF gene is accessible under the human gene nomenclature HGNC:1033 and shall comprise also its paralogs and orthologs. The NT-4 gene is accessible under the human gene nomenclature HGNC:8024, and shall comprise also its paralogs and orthologs (see http://www.genenames.org/).

In one further alternative embodiment of the invention the binding domain which specifically binds to the target cell is capable to bind to an expression product of a TrkA gene, preferably the NTRK1 gene (also referred to herein as “TrkA gene”), in the target cell. This embodiment is useful in the context of pain, preferably nociceptive and most importantly inflammatory pain. The expression product of the TrkA gene is a TrkA protein or a TrkA RNA, preferably TrkA mRNA. For example, the compound that is capable to bind to an expression product of the TrkA gene comprises a TrkA-ligand or an anti-TrkA-antibody or anti-TrkA-T cell receptor (TCR), or chimeric antigen receptor (CAR); or wherein the compound comprises a nucleic acid having a nucleic acid sequence that is complementary to, or can under stringent conditions hybridize to, an mRNA produced by the NTRK2 locus. Specific examples include a protein binding to the TrkA receptor and preferably is nerve growth factor (NGF). More preferred is a mutant variant of an NGF protein, such as a human NGF, wherein the mutations causes a loss of or reduction of neuronal signaling compared to the wild-type version of NGF, but wherein the binding to the target receptor is still maintained, possibly reduced. Most preferably an NGF is mutated at amino acid position R121, or the respective homologous position in a non-human NGF. Preferably the NGF is a NGF^{R121W} mutant.

In another aspect there is provided a conjugate compound that is capable of binding to TrkA, comprising a binding fragment of a TrkA ligand or a TrkA ligand, and which is conjugated to a cytotoxic agent and/or label. The conjugate compound according to this aspect is preferred in some embodiments, when the binding fragment of a TrkA ligand comprises the amino acid sequence of NGF and wherein the label or cytotoxic agent is a photosensitizing agent.

Preferably the binding fragment of a TrkA ligand comprises the amino acid sequence of NGF, or an amino acid sequence that is at least 70%, preferably 80%, 90%, or most preferably 95% identical to the amino acid sequence of NGF, preferably human NGF; and wherein the label or cytotoxic agent is a photosensitizing agent. The amino acid sequence of human NGF is the amino acid sequences of a protein expressed by the NGF gene. The NGF gene is accessible under the human gene nomenclature HGNC:7808 and shall comprise also its paralogs and orthologs (see http://www.genenames.org/). The human beta nerve growth factor amino acid sequence is also provided herein as SEQ ID NO: 2.

In another aspect of the invention there is provided a conjugate compound, comprising a binding fragment of a pruritogen, and which is conjugated to a cytotoxic agent and/or label. Preferably in some embodiments, wherein the binding fragment of a pruritogen comprises a binding fragment of an IL31RA/OSMR ligand or binding molecule. Preferably the ligand or fragment thereof comprises the amino acid sequence of IL31 or mutant IL31, or an amino acid sequence that is at least 70%, preferably 80%, 90%, or most preferably 95% identical to the amino acid sequence of IL31, preferably human IL31 and wherein the label or cytotoxic agent is a photosensitizing agent.

The IL31 gene is accessible under the human gene nomenclature HGNC:19372 and shall comprise also its paralogs and orthologs (see http://www.genenames.org/). The human protein sequence of IL31 is derivable from the Uniprot database under the accession number UniProtKB: Q6EBC2, in the version of the database of May 10, 2017.

### Diseases, Treatments and Pharmaceutical Compositions

The methods and compounds of the invention are preferably for use in the prophylaxis or treatment of a disease, or symptoms of a disease in a subject. A subject in context of the invention shall refer to an animal, preferably a mammal such as a mouse, dog, cat cow, monkey, ape, horse, rabbit, guinea pig, or human, and preferably is a human. The subject in preferred embodiments suffers from a pathological neurological sensation, as mentioned before, such as pathological touch sensation, pain, such as neuropathic pain or, preferably, inflammatory pain, or itch.

In some preferred embodiments of the invention, the herein described methods and compounds are for use in the prophylaxis or treatment of itch, or pathological itch, in the subject.

Itch is preferably itch associated with inflammatory skin reactions or diseases, or wherein the itch is not associated with inflammatory skin reactions or diseases such as pruritus associated with primary biliary cirrhosis, chronic renal failure/renal dialysis, abnormal blood pressure, thyroid gland malfunction, aging, cancer, anemia, a parasitic disease, a psycho-neurologic disease, a drug-induced disease and/or pregnancy, or pruritus induced by a pruritogen such as histamine, or wherein the itch is associated with chronic prurigo.

In other preferred embodiments of the invention the disease is an itch associated disease, such as atopic dermatitis, eczema and psoriasis.

In context of the herein disclosed methods and compounds for treatment, the invention provides that this treatment inhibits acute scratching of the subject.

In another embodiment of the invention there is also provided that the disease is pain, preferably neuropathic pain, and most preferably mechanical allodynia in the target body surface area of the subject.

The treatment in accordance to the invention involves in some embodiments a method wherein in a first step the conjugate compound comprising the binding domain and a photosensitive cytotoxin/inhibition moiety, or a moiety impairing otherwise neuronal function, is administered to a subject suffering from, or at danger of developing, a pathological neurological sensation as described (in particular pain or itch) in the target body surface area; and comprising a second step of illuminating said target body surface area with an appropriate excitation light in an amount to effectively activate said photosensitive cytotoxin group, or moiety impairing otherwise neuronal function, and to thereby induce neuronal retraction and/or inactivation/inhibition. In this context the term “body surface area” refers to a body surface, e.g. a skin area, where a patient suffers from, or is at danger to suffer from, the pathological neurological sensation.

The administration of the conjugate compound of the invention in this context may be performed systemically or locally to the targeted body surface area, for example by using injection or topical administration, or any other route known to the skilled artisan.

The term “systemic administration” or “systemically administering” means to denote administration through a route in which said agent inflicts a systemic effect. Systemic administration may typically be orally (including enteral or intragastric administration). However, other systemic administration routes are also possible, including, but not limited to, parenteral (e.g. intravenous, intraperitoneal, sub-dermal or intramuscular), nasal (e.g. via a nasal spray), in the form of an inhaled spray, transdermal delivery. A person versed in the art of formulating ingredients for system administration will be able to design a formulation on the basis of relevant pharmacokinetic and pharmacological considerations.

The term “local administration” used herein refers to administration at or near a specific site. Such an administration can be intradermal, subcutaneous or topical. Suitable pharmaceutical compositions for local administration may, for example, comprise eye/ear/nose drops, creams/ointments for dermal/ophthalmic application, sprays, aerosols, powders for insufflation, injections, inhalation, solutions/suspensions for nebulisation and the like.

It is one preferred embodiment of the invention that in context of medical treatments as disclosed herein, or compounds for use in such methods, that the conjugate compound in step (b) of the method of the first aspect is administered locally or systemically to the subject. For example, a local administration is a local administration of the conjugate compound at the target body surface area of the subject, for example, wherein the local administration is a subcutaneous injection, or topical administration, such as by applying a cream, ointment, salve, or other topical formulations. The target body surface area is an area where the—as described also above—the neurological sensation is perceived fully or in part.

It is preferred in some embodiments or aspects that the conjugate compound of the invention is administered in the form of a pharmaceutical composition comprising the conjugate compound together with a pharmaceutically acceptable salt or excipient.

In yet another aspect of the invention pertains to a pharmaceutical composition comprising a conjugate compound as described herein before, together with a pharmaceutically acceptable carrier and/or excipient.

In some embodiments, the subject pharmaceutical compositions of the present invention will incorporate the substance or substances to be delivered in an amount sufficient to deliver to a patient a therapeutically effective amount of an incorporated therapeutic agent, preferably the conjugate compound, or other material as part of a prophylactic or therapeutic treatment. The desired concentration of the conjugate compound as active agent will depend on absorption, inactivation, and excretion rates of the drug as well as the delivery rate of the conjugate compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of the subject conjugate compound (the active pharmaceutical ingredient—API) may be determined by reference to the plasma concentrations of the agent. For example, the maximum plasma concentration (Cmax) and the area under the plasma concentration-time curve from time o to infinity (AUC (0-4)) may be used. Dosages for the present invention include those that produce the above values for Cmax and AUC (0-4) and other dosages resulting in larger or smaller values for those parameters.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents of the invention employed in the pharmaceutical composition at levels lower than that required in order to achieve the desired therapeutic effect and gradually increase the dosage until the desired effect is achieved.

In addition, the invention shall also pertain to the following items:

Item 1: A compound for use in a method for treating mechanical allodynia in a subject suffering from neuropathic pain, the method comprising a step of inactivating one or more peripheral sensory neuron(s) that express Tropomyosin receptor kinase B (TrkB).

Item 2: The compound for use according to item 1, wherein the compound is capable to bind to an expression product of the TrkB gene.

Item 3: The compound for use according to item 2, wherein the expression product of the TrkB gene is a TrkB protein or a TrkB RNA, preferably TrkB mRNA.

Item 4: The compound for use according to any of items 1 to 3, wherein inactivating one or more peripheral sensory neuron(s) that express Tropomyosin receptor kinase B (TrkB), comprises inducing cytotoxicity in one or more peripheral sensory neuron(s) that express TrkB.

Item 5: The compound for use according to any of items 2 to 4, wherein the compound that is capable to bind to an expression product of the TrkB gene comprises a TrkB-ligand or an anti-TrkB-antibody or anti-TrkB-T cell receptor (TCR); or wherein the compound comprises a nucleic acid having a nucleic acid sequence that is complementary to, or can under stringent conditions hybridize to, an mRNA produced by the TrkB locus.

Item 6: The compound for use according to item 3, wherein the TrkB ligand is a protein binding to the TrkB/p75 receptor complex, and preferably is selected from Brain-derived neurotrophic factor (BDNF) or Neurotrophin 4 (NT-4).

Item 7: The compound for use according to any of items 2 to 6, wherein the compound that is capable to bind to TrkB is conjugated to a functional moiety.

Item 8: The compound for use according to item 7, wherein said functional moiety is a label and/or a cytotoxin.

Item 9: The compound for use according to item 8, wherein the label is a photosensitizing agent, such as phthalocyanine IRDye®700DX or a derivative thereof, such as benzylguanine modified phthalocyanine IRDye®700DX.

Item 10: The compound for use according to any of items 1 to 9, wherein the one or more peripheral sensory neuron(s) is a myelinated neuron that innervates hair follicles.

Item 11: The compound for use according to items 1 to 10, wherein the treatment essentially only alleviates mechanical allodynia and no other forms of hypersensitivity or pain in a subject.

Item 12: The compound for use according to items 1 to 11, wherein in a first step a compound binding to TrkB and which is conjugated to a photosensitive cytotoxin group is administered to a subject suffering from, or at danger of developing, mechanical allodynia in a target body surface area, and comprising a second step of irradiating said target body surface area with an appropriate excitation light in an amount to effectively activate said photosensitive cytotoxin group and to induce neuronal retraction and inactivation.

Item 13: The compound for use according to item 12, wherein the compound binding to TrkB is a TrkB ligand, or an anti-TrkB antibody, or anti-TrkB TCR.

Item 14: The compound for use according to item 12 or 13, wherein the photosensitive cytotoxin group is a phthalocyanine dye IRDye® 700DX, or a derivative thereof, such as a benzylguanine modified derivative.

Item 15: The compound for use for use in a method of treatment of mechanical allodynia in a subject suffering from neuropathic pain, wherein the compound is capable to selectively and/or specifically bind to an expression product of the TrkB gene.

Item 16: The compound for use according to item 15, wherein the treatment comprises a method according to any of items 1 to 14.

Item 17: A pharmaceutical composition for use according to item 15 or 16, comprising a compound is capable to selectively and/or specifically bind to an expression product of the TrkB gene and a pharmaceutically acceptable carrier and/or excipient.

Item 18: A method for identifying a peripheral sensory neuron mediating mechanical allodynia, the method comprising a step of determining the presence or absence of an expression product of the TrkB gene in a peripheral sensory neuron, wherein the presence of an expression product of the TrkB gene in the peripheral sensory neuron indicates that the peripheral sensory neuron mediates mechanical allodynia.

Item 19: A method of stratifying a subject suffering from mechanical allodynia into a group of subjects which benefit from a method according to any of items 1 to 14, comprising the step of determining the presence or absence of an expression product of the TrkB gene in a peripheral sensory neuron of the subject, wherein the presence of an expression product of the TrkB gene in the peripheral sensory neuron of the subject indicates that the subject will benefit from a treatment according to any of items 1 to 14.

The following figures, sequences, and examples merely serve to illustrate the invention and should not be construed to restrict the scope of the invention to the particular embodiments of the invention described in the examples. All references as cited herein are hereby incorporated in their entirety by reference.

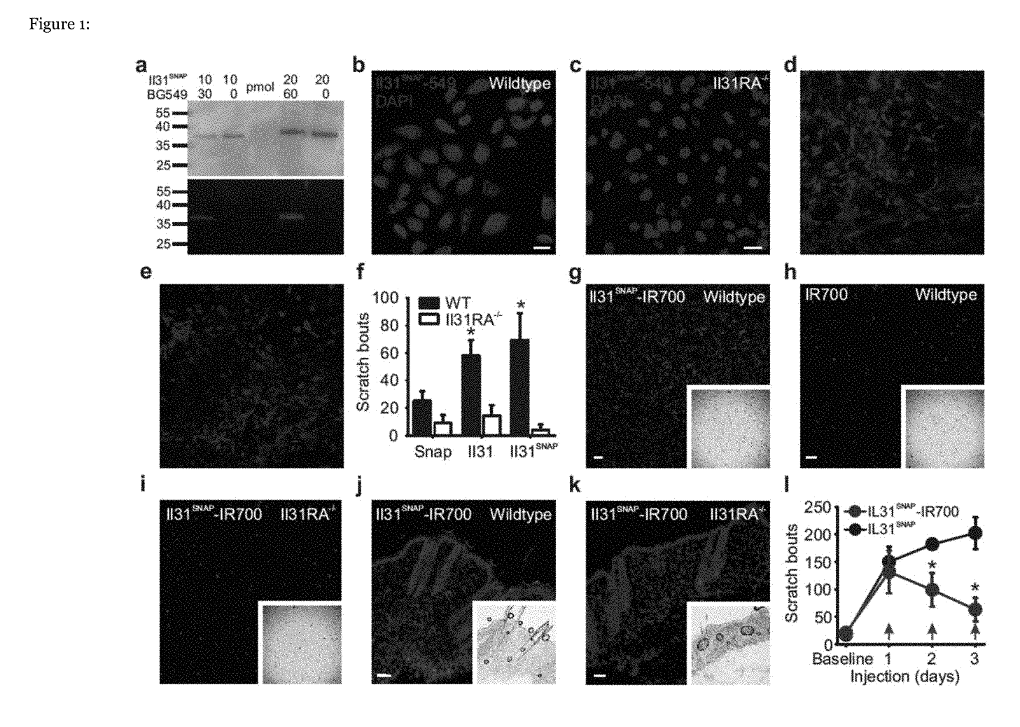

FIG. 1: IL31^{SNAP} labelling and photoablation. (a) Representative Coomassie (upper panel) and fluorescence (lower panel) gel showing the binding of IL31^{SNAP} with the fluorescent substrate BG549 at a 1:3 molar ratio. First and fourth lanes represent the binding of 10 and 20 pmol IL31^{SNAP} respectively, with BG549-Second and last lanes represent the protein IL31^{SNAP} alone, 10 and 20 pmol respectively. (b) Primary keratinocyte culture from wild type and (c) IL31RA^{-/-} mice labelled with 1 μM IL31^{SNAP} coupled with 3 μM BG549 (in red). Nuclei were stained with Dapi (in blue). Scale bar 20 μm. (d) Representative back skin cryosection (25 μm) of wild type and (e) IL31RA^{-/-} mice injected with 5 μM IL31^{SNAP} coupled with 15 μM BG549 (in red) and Dapi for nuclear staining (in blue). Scale bar 50 μm. (f) Scratching evoked by intradermal injection of 5 μM SNAP, IL31 or IL31^{SNAP} in wild type (n=4) and IL31RA^{-/-} mice (n=4). Error bars indicate SEM. *p<0.05 (t-test). (g, h, i) Propidium Iodide staining to assess cell death (in red) 24 hours after photoablation performed on primary wild type keratinocytes labelled with 1 μM IL31^{SNAP}+3 μM BGIR700 (g), with 3 μM IR700 only (h), or IL31RA^{-/-} keratinocytes labelled with 1 μM IL31^{SNAP}+3 μM BGIR700 (i). Insets represent the brightfield images of the stained cells. Scale bar 50 μm. (j, k) TUNEL assay staining to assess apoptosis (in red) after 3 consecutive days of photoablation performed on the back skin of wild type (j) and IL31RA^{-/-} (k) mice injected with 5 μM IL31SNAP+15 μM BGIR700. Insets represent the brightfield image of the same skin area. Scale bar 50 μm. (l) Scratching behavior evoked by 3 days of injection with 5 μM IL31^{SNAP} (black line) and 5 μM IL31^{SNAP}+15 μM IR700 (red line). Baseline refers to spontaneous scratching before the first injection. Number of scratch bouts was counted over a 30 minutes recording time. *p<0.05 (One-Way Anova).

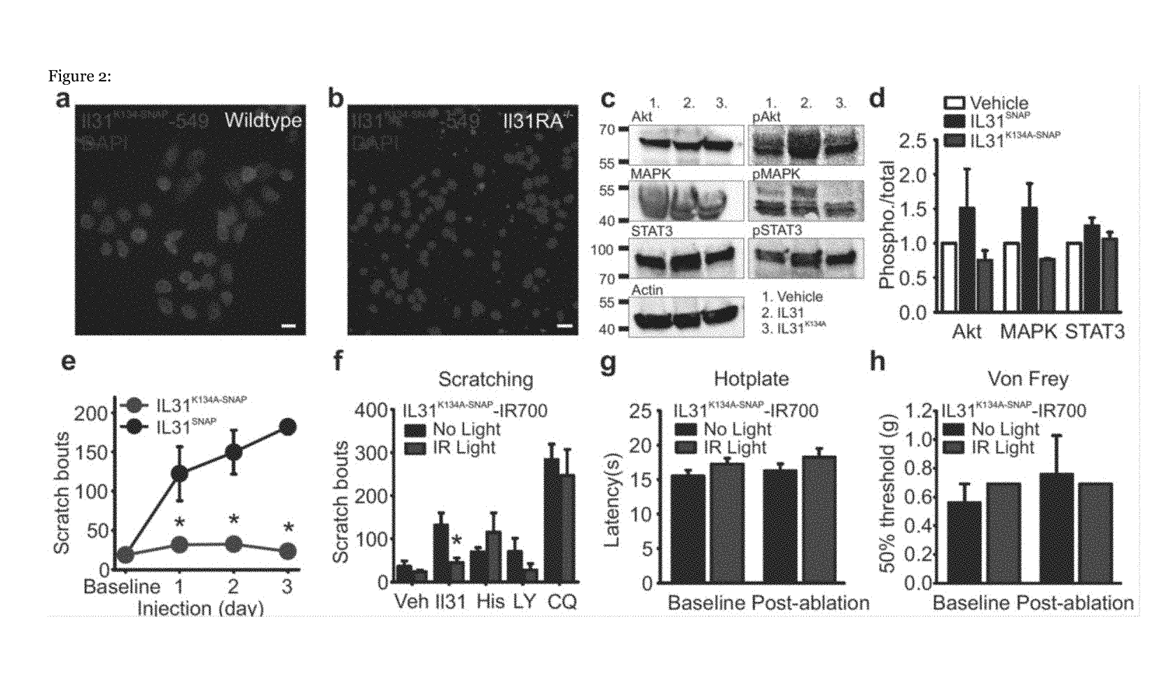

FIG. 2: Functional analysis of IL31^{K138A-SNAP}. (a) Primary keratinocyte cultures from wild type and (b) IL31iRA^{-/-} mice labelled with 1 μM IL31^{K138A-SNAP}+3 μM BG549 (in red). Nuclei were stained with DAPI (in blue). Scale bar 20 μm. (c) Representative western blots showing the expression level of AKT, phospho AKT, MAPK, phospho-MAPK, STAT3, phospho STAT3 and Actin (loading control) in skin injected with vehicle (PBS, lane i), 5 μM IL31^{SNAP} (lane 2) and 5 μM IL31^{K138A-SNAP} (lane 3). (d) Levels of each protein were expressed as the ratio between the phosphorylated form and the total counterpart and then normalized to the vehicle-treated sample. (e) Scratching response evoked by 3 consecutive days of injection of 5 μM IL31^{SNAP} (black line) and 5 μM IL31^{K138A-SNAP} (red line). Baseline refers to spontaneous scratching before the first injection. Number of scratching bouts was counted over a 30 minutes recording time. Error bars indicate SEM. *p<0.05 (One-Way Anova). (f) Scratching response evoked by the injection of vehicle (PBS, N=4) and different pruritogens (5 μM IL31, 10 mM Histamine, 1 mM LY344864, and 12.5 mM Chloroquine CQ) after mice were injected for 3 consecutive days with 5 μM IL31^{K138A-SNAP}+15 μM BGIR700, with (red bars, n=5) and without (black bars, n=4) near IR illumination. The number of scratching bouts was counted over a 30 minute recording time. Error bars indicate SEM. *p<0.05 (t-Test). (g) Thermal sensation was evaluated using the Hot Plate test after 3 days of injection of 5 μM IL31^{K138A-SNAP}+15 μM BGIR700 into the hind paw of the mice, with (red bars, n=4) and without (black bars, n=4) near IR illumination. Baseline refers to the thermal latency before the first injection was performed. Bar graphs represent the latency expressed in seconds of the paw withdrawal in response to heat. Error bars indicate SEM. (h) Mechanical sensation was evaluated using the Von Frey test after 3 days of injection of 5μM IL31^{K138A-SNAP}+15 μM BGIR700 into the hind paw of the mice, with (red bars, n=4) and without (black bars, n=4) near IR illumination. Baseline refers to the mechanical threshold before the first injection was performed. Bar graphs represent the force expressed in grams required to trigger a 50% response. Error bars indicate SEM.

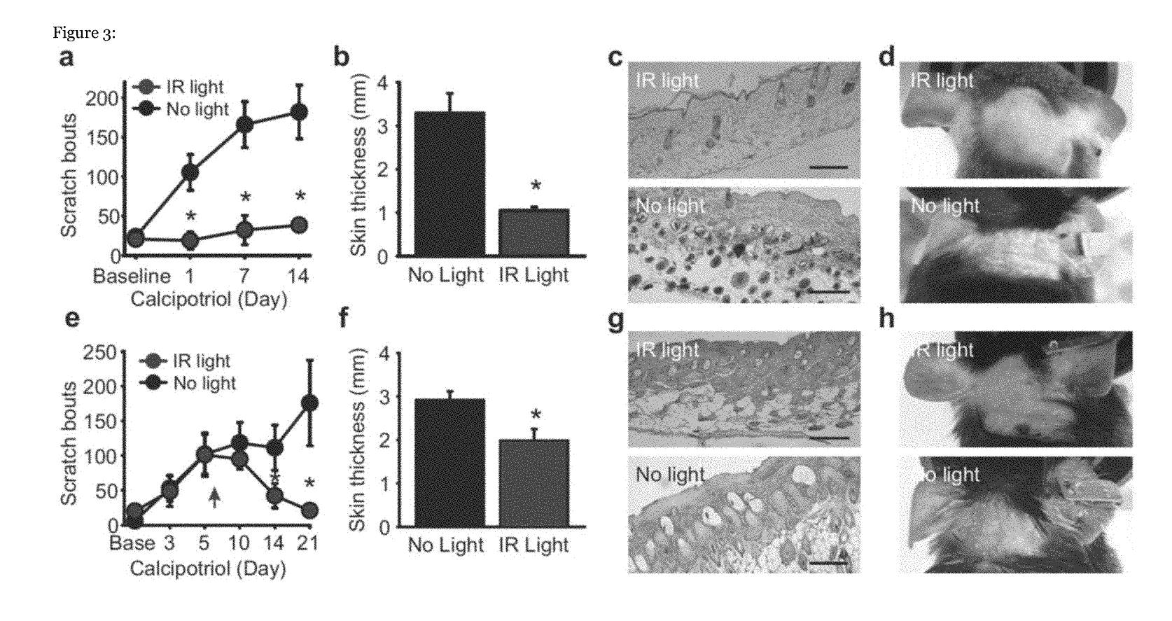

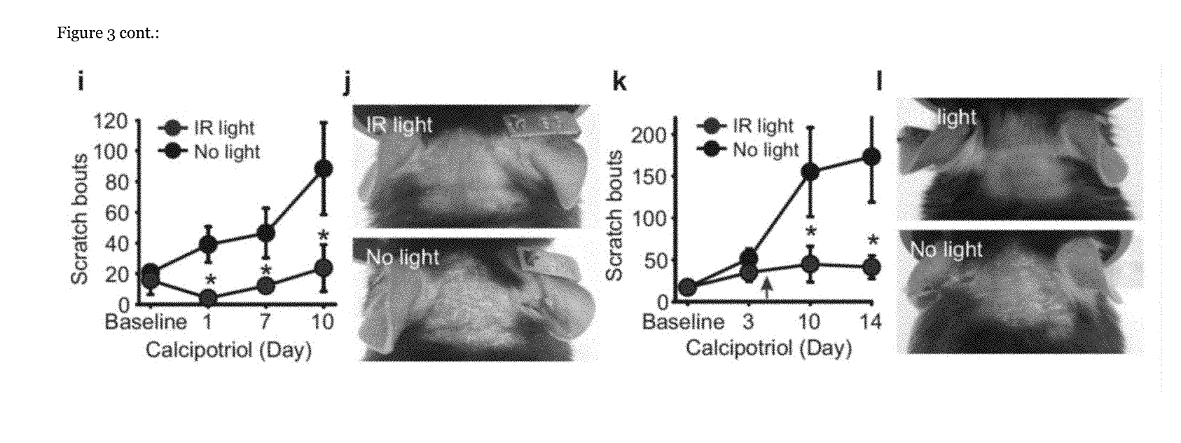

FIG. 3: IL31 ^{K138A-SNAP} guided photoablation prevents and reverses symptoms of atopic dermatitis. (a-d) Prevention of atopic dermatitis-like symptoms. (a) Number of scratch bouts in response to 14 days of Calcipotriol treatment in mice pre-injected for 3 consecutively days with 5 μM IL31^{K138A-SNAP}+15 μM BGIR700, with (red line, n=4) or without (black line, N=4) near IR illumination. The number of scratch bouts was counted over a 30-minute recording time. Baseline refers as spontaneous scratching before injections were performed. Error bars indicate SEM. * p<0.05 (One-Way Anova). (b) Skin thickness expressed in millimeter (mm) and measured at day 14 of Calcipotriol treatment in mice injected with IL31^{K138A-SNAP}+IR700, with (red bar, n=4) and without (black bar, n=4) near IR illumination. Error bars indicate SEM. *p<0.05 (t-Test). (c) Hematoxylin & Eosin staining of 6 μm-paraffin sections of back skin collected after 14 days of Calcipotriol treatment showing difference in skin histology (epidermal thickness and dermal infiltration of eosinophilic material) between mice treated with IL31^{K138A-SNAP}+IR700, with (Top panel) IR light and without (Lower panel, No light) near IR illumination. Scale bars 200 μm. (d) Representative skin pictures of mice after 14 days of Calcipotriol treatment treated with IL31^{K138A-SNAP}+IR700 with near IR illumination (Top panel) and without (Lower panel) (e-h) Rescue of atopic dermatitis symptoms after treatment with IL31^{K138A-SNAP} at days 6-8 of Calcipotriol application. (e) Scratching bouts with (red line, n=6) and without (black line, N=6) near IR illumination. Error bars indicate SEM. *p<0.05 (One-Way Anova;). (f) Skin thickness at day 21 of Calcipotriol treatment (n=6 both groups). Error bars indicate SEM. *p<0.05. (g) Hematoxylin & Eosin staining of 6 μm-paraffin sections of back skin after 21 days of Calcipotriol treatment. Scale bars 200 μm. (h) Representative skin images at 21 days of Calcipotriol treatment. (i-j) Prevention of atopic dermatitis-like symptoms using topical delivery of IL31^{K138A-SNAP} (i) Scratching behavior evoked by Calcipotriol. Error bars indicate SEM. *p<0.05 (One-Way Anova). (j) Representative skin pictures of mice after 10 days of Calcipotriol application. (k-l) Rescue of atopic dermatitis symptoms using topical application of IL31^{K138A-SNAP} at day 5-7 of Calcipotriol applications. (k) Scratching behavior (red circles, n=7, black circles, n=8) for 3 consecutive days Error bars indicate SEM. *p<0.05 (One-Way Anova). (l) Representative skin pictures after 14 days of Calcipotriol application.

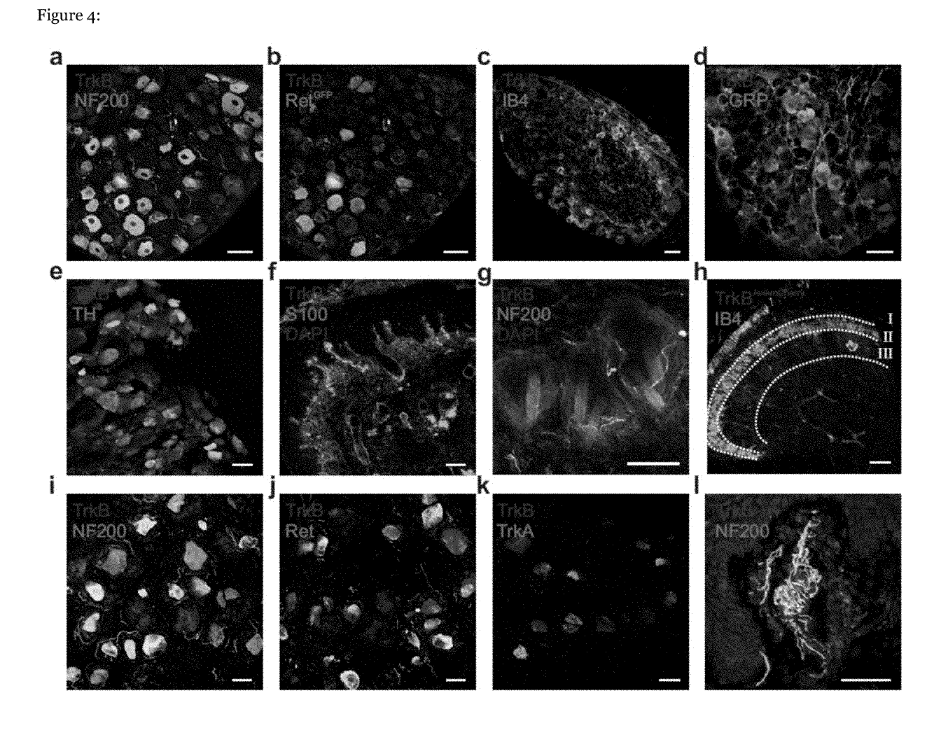

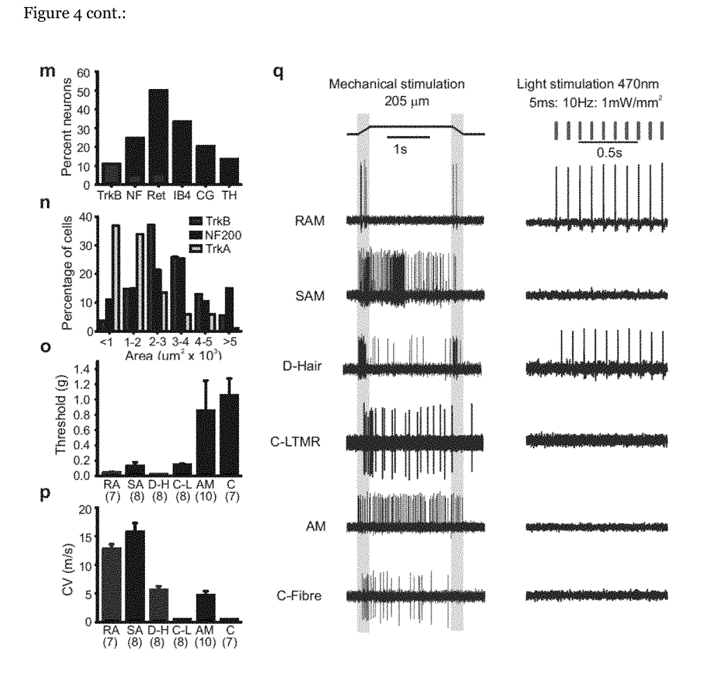

FIG. 4: TrkB positive sensory neurons are myelinated low threshold mechanoreceptors. (a-e) Double immunofluorescence of DRG sections from TrkBCreERT2::Rosa26RFP mice with (a) NF200, (b) Ret, visualized using TrkBCreERT2::Rosa26RFP::RetEGFP triple transgenic mice, (c) IB4, (d) CGRP, and (e) TH. (f) Section from the glabrous skin of TrkBCreERT2::Rosa26ChR2YFP (red) stained with anti-S100 a marker for Meissner's corpuscles (green) and DAPI (blue) showing TrkB+ innervation. (g) TrkB+ lanceolate endings in a section of the back hairy skin of TrkBCreERT2::Rosa26SnapCaaX labeled with Snap Cell TMRstar (red), NF200 (green) and DAPI (blue). (h) Section through the lumbar spinal cord of TrkBCreERT2::AvilmCherry mice stained with IB4. (i-k) Double immunofluorescence of human DRG sections stained with antibodies against TrkB and (i) NF200, (j) Ret and (k) TrkA. (l) Section from human glabrous skin stained with antibodies against TrkB (red) and NF200 (green), and DAPI (blue). (m) Quantification of staining on mouse DRG sections; TrkB+ cells account for ˜10% of all DRG neurons and all co-express NF200 (NF) or NF200+ReteGFP, while they are negative for IB4, CGRP (CG) and TH. (n) Size distribution for human DRG neurons expressing TrkB, NF200 and TrkA. (o-q) in-vitro skin nerve preparation from TrkBCreERT2::Rosa26ChR2 mice showing (o) the minimal force required to elicit an action potential in the indicated fibre type, (p) the conduction velocities of the fibre types and (q) representative responses to 10 Hz stimulation with blue light. Red bar represents TrkB+ afferents, n number indicated in brackets. Scale bars, A-E and H 50 μm, F, G and I-L 40 μm. Error bars represent SEM. TrkB positive sensory neurons are myelinated low threshold mechanoreceptors.

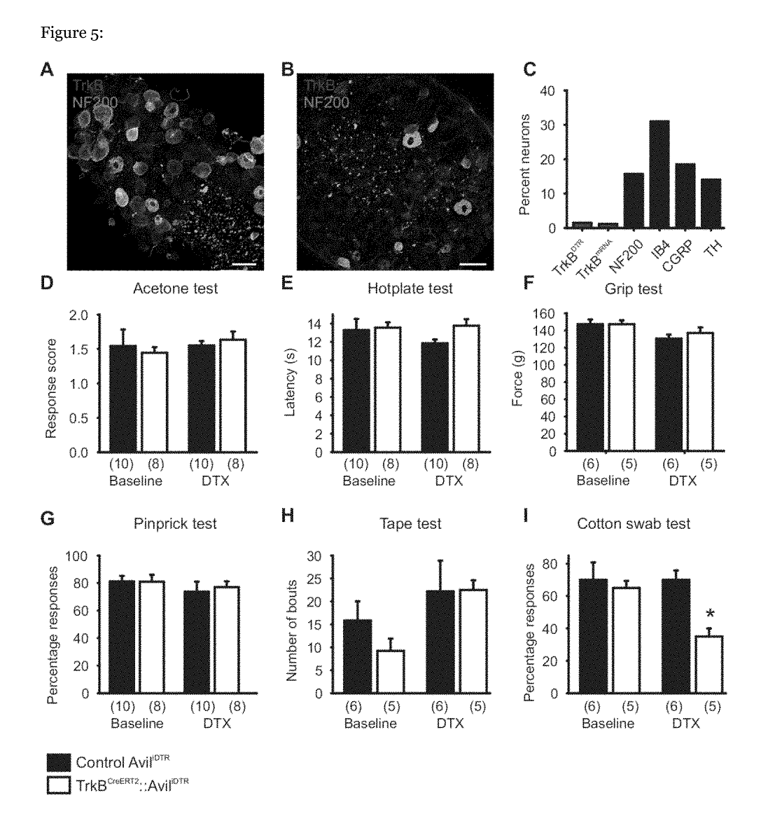

FIG. 5: Diphtheria toxin mediated ablation of TrkB+ sensory neurons. Immunostaining of DRG sections of TrkB^{CreERT2}::Avil^{iDTR} mice with an antibody against the diphtheria toxin receptor (red) from (A) untreated mice and (B) after i.p injections of diphtheria toxin. (C) Quantification of DRG sections indicating a ˜90% decrease in TrkB^{DTR} and Trke^{mRNA} cells after ablation and ˜40% reduction in NF200⁺ neurons without affecting other subpopulations. (D-J) Behavioral responses in littermate control mice (Avil^{iDTR}, black bars) and TrkB^{CreERT2}::Avil^{iDTR} mice (white bars) showing no differences in responses before and after ablation in the (D) acetone drop test (t-test; p>0.05), (E) hot plate test (t-test; p>0.05), (F) grip test (t-test; p>0.05), (G) pin-prick test (t-test; p>0.05), (H) tape test (t-test; p>0.05). (I) Ablated mice show a reduction in sensitivities to cotton swab (t-test, p<0.001). Scale bars in A, B 50 μm, error bars indicate SEM.

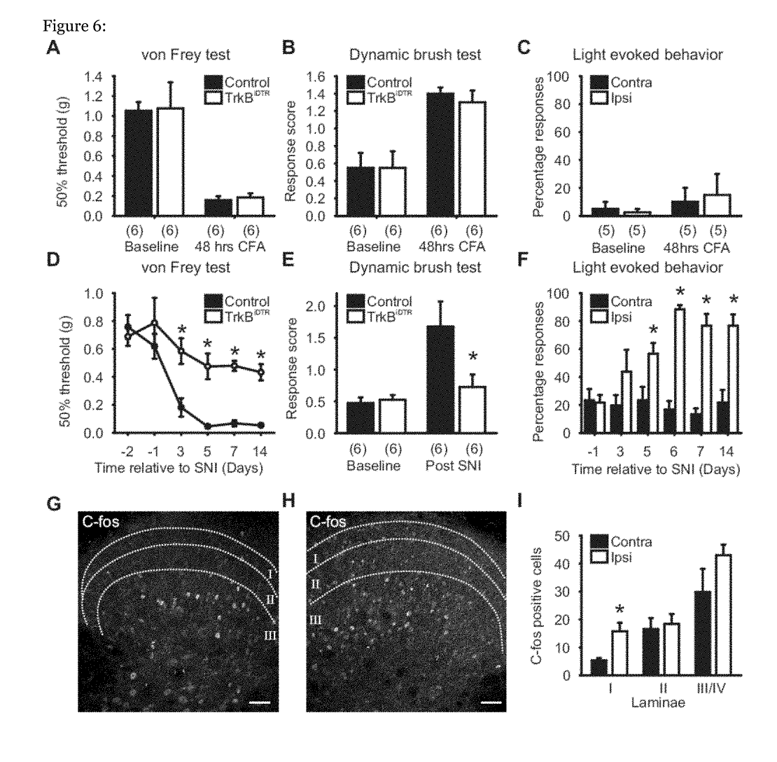

FIG. 6: TrkB+ neurons are necessary and sufficient to convey mechanical allodynia after nerve injury. Mechanical hypersensitivity in both control Avil^{iDTR} (black bar) and TrkB^{CreERT2}::Avil^{iDTR} (white bar) mice 48 hours after CFA injections as measured by (A) von Frey filaments (t-test, p>0.05) and (B) dynamic brush stimuli (t-test; p>0.05). All mice received two diphtheria toxin injections 7 days and to days before CFA treatment. (C) Paw withdrawal frequencies in contralateral (black bar) and CFA injected ipsilateral (white bar) paw of TrkB^{CreERT2}::Rosa26^{ChR2} mice upon stimulation with 473 nm blue light shows no significant difference under baseline conditions and 48 hours after CFA injection (Mann-Whitney test; p>0.05). (D) von-Frey mechanical thresholds indicating that ablation of TrkB+ neurons abolished the development of mechanical allodynia after SNI in TrkB^{CreERT2}::Avil^{iDTR} mice (white circles) as compared to Avil^{iDTR} controls (black circles) (n=7 for both sets, Two-way RM ANOVA; p<0.001 followed by a Bonferroni post-hoc test). (E) Reduced dynamic brush allodynia in ablated TrkB^{CreERT2}::Avil^{iDTR} mice (white bar) as compared to littermate controls (black bar; t-test p<0.05). (F) Nociceptive behavior evoked by optogenetic stimulation of ipsilateral (black bars) and contralateral (white bar) paw of TrkB^{CreERT2}::Rosa26^{ChR2} mice after SNI (Two-way RM ANOVA; p<0.001). (G-H) Cross section of the lumbar spinal cord from TrkB^{CreERT2}::Rosa26^{ChR2} mice labelled for c-fos after 1 minute exposure to a 15 Hz blue light 7 days post SNI (G) Represents the contralateral uninjured and (H) the injured ipsilateral dorsal horn. (I) shows quantification of the number of c-fos positive cells in each lamina of the lumbar spinal cord within a 40 μm section (black bar contralateral, white bar ipsilateral). Error bars indicate SEM. Scale bars in G and H, 40 μm.

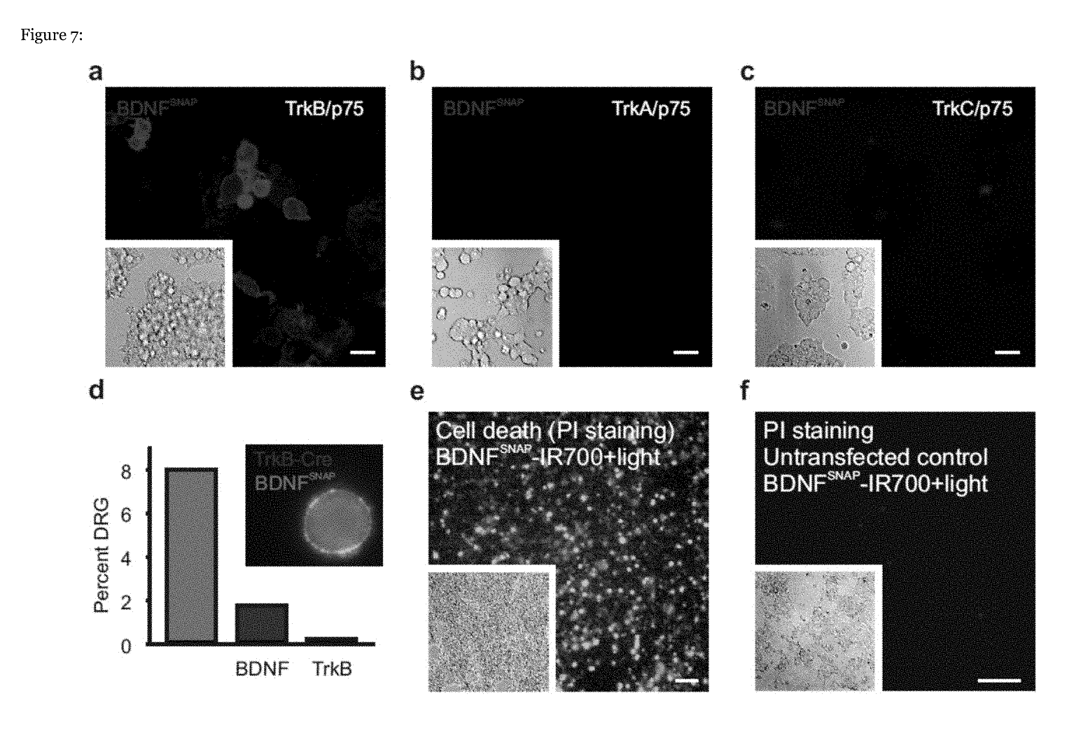

FIG. 7: BDNF^{SNAP} labelling and IR700 mediated photoablation in vitro. (a-c) BDNF^{SNAP} labeling of HEK293T cells transfected with (a) TrkB/p75NTR, (b) TrkA/p75NTR, or (c) TrkC/p75NTR. (d) Labeling (inset) and quantification of dissociated DRG from TrkB^{CreERT2}::Rosa26^{RFP} mice with BDNF^{SNAP} shows substantial overlap of BDNF^{SNAP} binding to TrkB+ cells. (e)

Staining of HEK293T cells transfected with TrkB/p75NTR with propidium iodide 24 hours after treatment with BDNFSNAP-IR700 and near infrared illumination. (f) Staining of mock transfected HEK293T cells with propidium iodide 24 hours after photoablation following treatment with BDNFSNAP-IR700. Scale bars 50 μm.

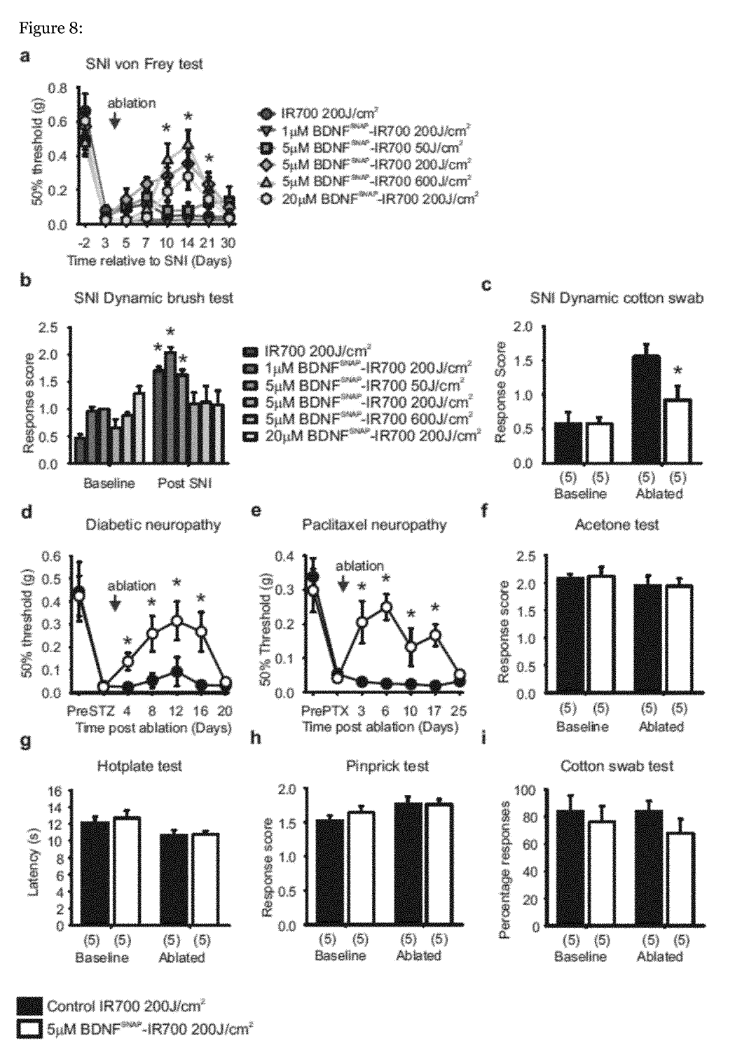

FIG. 8: Optopharmacological targeting of TrkB+ neurons with BDNF^{SNAP}. (a-b) BDNF^{SNAP}-IR700 mediated photoablation of the paw of SNI mice results in a dose dependent reversal of mechanical hypersensitivity as assayed with von Frey filaments (a) (n=10, two-way RM ANOVA; p<0.05 followed by a Bonferroni post-hoc test) and (b) dynamic brush stimuli (t-test; p<0.05). (c) Hypersensitivity to cotton swab is also reversed by photoablation (t-test: p<0.05). (d) BDNF^{SNAP}-IR700 mediated photoablation reverses mechanical allodynia in the streptozotocin (STZ) model of diabetic neuropathy (n=5, two-way RM ANOVA; p<0.05 followed by a Bonferroni post-hoc test. Open circles; 5 μM BDNF^{SNAP}-IR700 at 200 J/cm², closed circles, 5 μM IR700 at 200 J/cm²). (e) BDNF^{SNAP}-IR700 mediated photoablation reverses mechanical allodynia in the paclitaxel (PTX) model of chemotherapy induced neuropathy (n=5, two-way RM ANOVA; p<0.05 followed by a Bonferroni post-hoc test. Open circles; 5 μM BDNF^{SNAP}-IR700 at 200 J/cm², closed circles, 5 μM IR700 at 200 J/cm²). (f-i) BDNF^{SNAP}-IR700 mediated photoablation in the paw does not affect baseline sensory behavior responses to (f) acetone drop test (t-test; p>0.05), (g) hot plate test (t-test; p>0.05), (h) pin-prick test (t-test; p>0.05) and (i) cotton swab test (t-test; p>0.05). White bars 5 μM BDNF^{SNAP}-IR700 at 200 J/cm², black bars 5 μM IR700 at 200 J/cm². Baseline indicates pre-ablation and pre-treatment. Error bars indicate SEM.

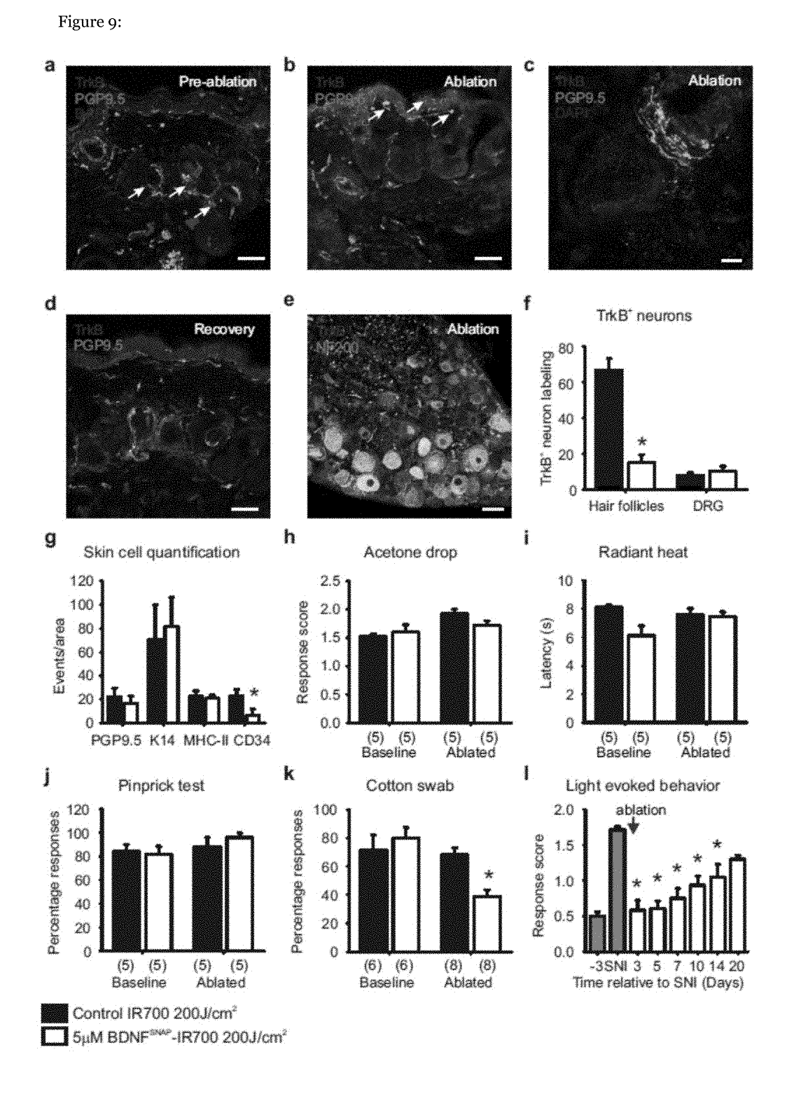

FIG. 9: BDNF^{SNAP}-IR700 photoablation promotes local retraction of TrkB+ afferents. (a-d) Substantial loss of TrkB^{CreERT2} positive afferents (red), but persistence of other fibers (green) upon BDNF^{SNAP}-IR700 mediated photoablation. (a) Innervation of paw hairy skin prior to ablation, arrows show lanceolate endings. (b) Loss of TrkB^{CreERT2} afferents after ablation, arrows show PGP9.5 fibers. (c) High magnification image of a hair follicle after ablation. Note the absence of TrkBCreERT2 fibers (red) but PGP9.5 positive circumferential and longitudinal lanceolate endings (green). (d) Reinnervation of skin by TrkB^{CreERT2} afferents at 24 days post ablation. (e) DRG section from photoablated TrkB^{CreERT2} mouse labelled for RFP (red) and NF200 (green). (f) Quantification of the proportion of hair follicle innervation and DRG neurons positive for TrkB following photoablation in the paw. (g) Quantification of loss of other cells types in the skin upon photoablation. (h-l) Behavioral sensitivity following BDNF^{SNAP}-IR700 mediated ablation in the sciatic nerve. (h) Acetone drop test (t-test; p>0.05), (i) radiant heat test (t-test; p>0.05), and (j) pin-prick test (t-test; p>0.05) are not altered by nerve photoablation. However sensitivity to (k) cotton swab (t-test; p<0.05) in control animals, and (1) light evoked behavior in TrkB^{CreERT2}::Rosa26^{ChR2} mice with SNI, are reduced by nerve photoablation (Two-way RM ANOVA; p<0.001). White bars 5 μM BDNF^{SNAP}-IR700 at 200 J/cm², black bars 5 μM IR700 at 200 J/cm². Baseline indicates pre-ablation and pre-treatment. Error bars indicate SEM. Scale bars a-d 40 μm, e 10 μm.

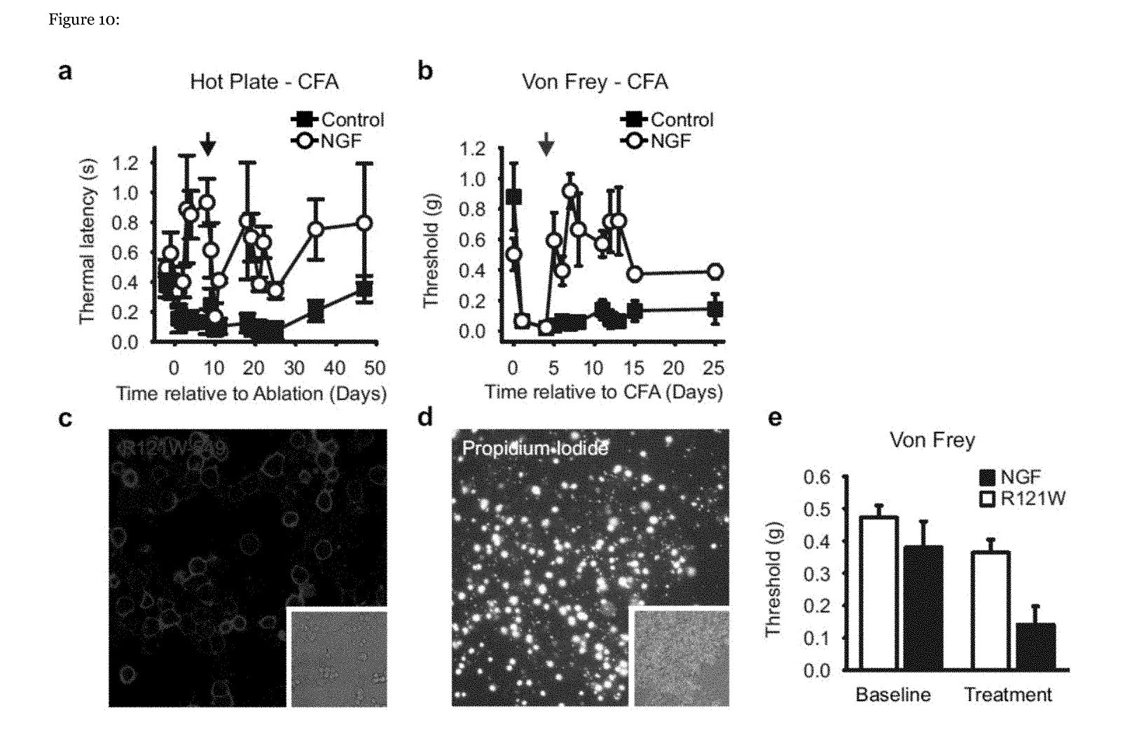

FIG. 10. NGF^{SNAP}-IR700 mediated photoablation and identification of a painless NGF mutant. (a) NGF^{SNAP}-IR700 mediated photoablation prevents thermal hyperalgesia in the CFA model of inflammatory pain. Blue arrow indicates CFA application. (b) NGF^{SNAP}-IR700 mediated photoablation reverses mechanical hypersensitivity following CFA injections. Red arrow indicates photoablation. (c) The NGFR^{121W-SNAP} mutant binds to cells expressing TrkA and p75. (d) Photoablation of HEK293 cells expressing TrkA and p75 upon application of NGF^{R121W-SNAP} and illumination. (e) Wildtype NGF^{SNAP} induces mechanical hypersensitivity when injected in the paw (black bars), while NGF^{R121W-SNAP} does not (white bars).

FIG. 11. NGFR^{121W-SNAP}-IR700 mediated photoablation to control acute and inflammatory pain. (a). Injection of NGFR^{121W-SNAP}-IR700 into the paw and subsequent near-IR illumination substantially increases paw withdrawal thresholds to von Frey filaments. (b) Nociceptive pin prick evoked responses are significantly reduced by NGFR^{121W-SNAP}-IR700 photoablation. (c) Non-nociceptive brush sensitivity is not altered by NGFR^{121W-SNAP}-IR700 mediated photoablation. (d) CFA induced thermal hyperalgesia is reversed by NGFR^{121W-SNAP}-IR700 photoablastion. (e) CFA induced mechanical hypersensitivity is reversed by NGFR^{121W-SNAP}-IR700 mediated photoablation. Red arrow indicates time of photoablation, control indicates injection without illumination.

SEQ ID NO: 1 shows wild type human IL31 amino
acid sequence:
MASHSGPSTSVLFLFCCLGGWLASHTLPVRLLRPSDDVQKIVEELQSL
SKMLLKDVEEEKGVLVSQNYTLPCLSPDAQPPNNIHSPAIRAYLKTIR
QLDNKSVIDEIIEHLDKLIFQDAPETNISVPTDTHECKRFILTISQQF
SECMDLALKSLTSGAQQATT
SEQ ID NO: 2 shows wild type human NGF amino
acid sequence:
MSMLFYTLITAFLIGIQAEPHSESNVPAGHTIPQAHWTKLQHSLDTAL
RRARSAPAAAIAARVAGQTRNITVDPRLFKKRRLRSPRVLFSTQPPRE
AADTQDLDFEVGGAAPFNRTHRSKRSSSHPIFHRGEFSVCDSVSVWVG
DKTTATDIKGKEVMVLGEVNINNSVFKQYFFETKCRDPNPVDSGCRGI
DSKHWNSYCTTTHTFVKALTMDGKQAAWRFIRIDTACVCVLSRKAVRR
A

### EXAMPLES

### I: ITCHING

### Example 1: Generation and Characterization of IL31^{SNAP}

The inventors produced recombinant IL31 with a C-terminal fusion of SNAP (IL31^{SNAP}) in E. Coli. Following purification and refolding from inclusion bodies, IL31^{SNAP} was efficiently labelled with BG derivatized fluorophores (FIG. 1a) indicating that the SNAP tag was successfully incorporated and correctly folded in the fusion protein. To determine whether IL31^{SNAP} was functional the inventors first performed binding studies in primary keratinocyte cultures. IL31^{SNAP} was labelled in vitro with BG-Surface549 and applied to keratinocytes from wildtype or IL31 Receptor A (IL31RA) knockout mice (IL31RA^{-/-). At a range of concentrations the inventors observed strong fluorescent signal internalized in wildtype keratinocytes that was not present in cells for IL}31RA^{-/-} mice (FIGS. 1b and c). The inventors further examined IL31^{SNAP} labelling in vivo by injecting IL31^{SNAP}-Surface549 intradermally into the back skin of wild type and IL31RA^{-/-} mice. Again, fluorescent signal was observed in cells in skin sections from wildtype mice but not from IL31RA^{-/-} mice (FIGS. 1d and e). Finally, the inventors determined whether IL31^{SNAP} was active by quantifying scratching behavior in mice upon intradermal injection. IL31^{SNAP} evoked robust scratching that was comparable in duration and intensity to native recombinant IL31 in wildtype mice. In IL31RA^{-/-} mice, IL31^{SNAP} and IL31 did not evoke scratching (FIG. 1f). Thus the IL31^{SNAP} retains the functional properties of native IL31.

### Example 2: IL31 Mediated Photoablation

To manipulate IL31 receptor expressing cells in vivo, the inventors reasoned that IL31^{SNAP} may allow for targeted photoablation of these cells through delivery of a photosensitizing agent. The inventors synthesized a benzylguanine modified derivative of the highly potent near-infrared photosensitizer IRDye®700DX phthalocyanine (IR700) and conjugated it in vitro to IL31^{SNAP} (20). Application of IL31^{SNAP}-IR700 to keratinocytes followed by 1 minute illumination provoked substantial cell death (FIG. 1g) that was not evident in keratinocytes only treated with IR700 (FIG. 1h) or in keratinocytes from IL31RA^{-/-} mice (FIG. 1i). The inventors further examined photosensitizer induced cell death in skin by injecting IL31^{SNAP}-IR700 and applying near infrared light to the skin. TUNEL positive apoptotic cells were observed throughout the epidermis and dermis of the illuminated area in wildtype mice (FIG. 1j), but largely absent in skin from IL31RA^{-/-} mice (FIG. 1k). The inventors next examined whether IL31^{SNAP}-IR700 mediated photoablation would impact upon IL31 evoked scratching behavior. Strikingly, a progressive decrease in scratching bouts was observed when IL31^{SNAP}-IR700 was injected for three consecutively days and the skin illuminated (FIG. 1l).

### Example 3: Generation and Characterization of a Non-Signaling IL31 Mutant

A conceptual problem of using IL31^{SNAP} therapeutically is that it in itself evokes itch. The inventors therefore sought to engineer IL31^{SNAP} to obtain a ligand that still binds to IL31 receptor complex but no longer promotes signaling. From a previous structure/function study (21) the inventors selected an IL31 point mutant IL31^{K138A} that was reported to exhibit reduced signaling in cells expressing IL31 receptors. The K138A mutation denotes the murine IL31 mutations. The corresponding mutation in the human IL31 protein is at position K134 in the human IL31 (SEQ ID NO: 1). The inventors generated a recombinant IL31^{K138A-SNAP} fusion protein, labelled it with BG-Surface549 and applied it to keratinocytes. Pronounced fluorescence was evident in cells from wildtype mice treated with fluorescent IL31^{K138A-SNAP} (FIG. 2a), at a similar concentration range to that observed with IL31^{SNAP} (Supplementary FIG. 2). Importantly, such signal was not present in IL31RA^{-/-} keratinocytes (FIG. 2b). The inventors further assessed cellular signaling pathways activated by IL31^{SNAP} and IL30^{K138A-SNAP} in the skin by examining levels of phosphorylated Akt, pMAPK and pSTAT3 which have all been previously implicated in IL31 downstream signaling (14, 21, 22). Mice were injected subcutaneously with IL31^{SNAP} and IL31^{K138A-SNAP} and skin harvested 1 hour later for immunoblot analysis. The inventors observed increased phosphorylation in each pathway in skin injected with IL31^{SNAP}, and this increase was absent in skin injected with IL311^{K138A-SNAP} (FIGS. 2c and d). As a final test for the functional activity of IL31^{K138A-SNAP}, the inventors assayed its capacity to provoke scratching behavior in mice. In contrast to IL31^{SNAP} which induced robust scratching when injected intradermal, IL31^{K138A-SNAP} did not evoke any scratching above baseline levels in mice (FIG. 3e). Thus the engineered ligand IL31^{K138A-SNAP} may offer a powerful means of targeting cells involved in itch without triggering itch in itself.

### Example 4: IL31^{K138A-SNAP}-IR700 Mediated Photoablation and Acute Itch

To characterize IL31^{K138A-SNAP} mediated photoablation in vivo, the inventors first tested its efficacy at alleviating IL31 provoked itch. Mice were treated for three consecutively days with IL31^{K138A-SNAP}-IR700 and the skin was illuminated with near-IR light. Strikingly, IL31-induced scratching behavior was abolished in these animals (FIG. 2f), and this persisted throughout an 8 week observation period (Supplementary FIG. 2l). In control mice that received an IL31^{K138A-SNAP}-IR700 injection but were not illuminated, the inventors observed no reduction in IL31-evoked scratching (FIG. 2f). The inventors next tested the effects of photoablation on scratching provoked by other acutely applied pruritogens. Intriguingly, IL31^{K138A-SNAP} guided photoablation had no significant effect on histamine, chloroquine or LY344864 (a serotonin 5-HT1F receptor agonist) evoked itch (FIG. 2f). Finally, to assess the specificity of photoablation to itch sensation, the inventors examined other sensory modalities after treatment. Using the hot plate test to assay thermal sensitivity (FIG. 2g), and calibrated von Frey filaments to measure mechanical sensitivity (FIG. 2h), the inventors observed no difference in response properties of treated mice, indicating that IL31 guided laser ablation is indeed a selective and effective means of disrupting the itch pathway.

### Example 5: IL31^{K138A-SNAP}-IR700 Mediated Photoablation and Chronic Inflammatory Itch

The inventors examined the effects of IL31^{K138A-SNAP}-IR700 ablation on inflammatory skin conditions using the well characterized Calcipotriol model of atopic dermatitis (23). To assess the effectiveness of treatment the inventors monitored three indicators of clinical progression; scratching behavior, skin integrity and skin histology. The inventors first determined whether pretreatment with IL31^{K138A-SNAP}-IR700 would abolish the development of the disease, and then investigated whether post-treatment, upon establishment of robust inflammation, could reverse symptoms.

As previously reported (M. Li et al., Topical vitamin D3 and low-calcemic analogs induce thymic stromal lymphopoietin in mouse keratinocytes and trigger an atopic dermatitis. Proc Natl Acad Sci USA 103, 11736-11741 (2006)), application of Calcipotriol to skin provoked a severe atopic dermatitis-like phenotype that was evident as a progressive increase in the number of spontaneous scratching bouts over time, distinct skin damage, and a thickening of the epidermis and cell infiltration (not shown). Injection of IL31^{K138A-SNAP}-IR700 and subsequent near IR illumination of the skin for 3 days prior to Calcipotriol application completely abolished the development of all indicators in this model. Thus scratching behavior remained at baseline levels (FIG. 3a), skin thickness and histological characteristics were not altered (FIGS. 3b and c), skin appeared healthy and typical features of dermatitis such as redness and scaling were entirely absent (FIG. 3d). To control for a pharmacological antagonistic effect of IL31^{K138A-SNAP} the inventors performed identical experiments in the absence of near IR illumination and observed the normal development of dermatitis-like symptoms (FIGS. 3a-d). Similarly, near IR light and IR700 alone were also ineffective at blocking the progression of the condition (not shown).

For IL31^{K138A}-guided photoablation to be developed as a clinical tool, it must also be effective in reversing already established skin inflammation. The inventors therefore treated mice with Calcipotriol for 7 days until severe symptoms were evident. IL31^{K138A-SNAP}-IR700 was then injected subcutaneously and near IR light applied to the skin for three consecutively days. Strikingly, the inventors observed a rescue of all disease indicators over the course of 1 week. Thus scratching behavior returned to baseline levels (FIG. 3e), and skin thickness (FIG. f), morphology (FIG. 3g)) and structure (FIG. 3h) became indistinguishable from healthy mice. Such profound reversal of dermatitis-like symptoms was not evident in control experiments where IL31^{K138A-SNAP} was applied without subsequent near IR illumination (FIG. 3e-h).

Finally, to further improve the clinical applicability of IL31^{K138A} guided photoablation, the inventors sought to develop a formulation that would allow for topical, pain-free application of ^{IL31K138A-SNAP}-IR700. The inventors selected a water-in-oil micro-emulsion preparation based upon previous evidence that this type of formulation can deliver high molecular weight proteins across the dermal barrier (R. Himes, S. Lee, K. McMenigall, G. J. Russell-Jones, Reduction in inflammation in the footpad of carrageenan treated mice following the topical administration of anti-TNF molecules formulated in a micro-emulsion. J Control Release 145, 210-213 (2010)). IL31^{K138A-SNAP}-IR700 was loaded into the aqueous phase of the micro-emulsion, applied topically and 20 minutes later, skin was illuminated with near IR light. Similar to subcutaneous delivery, topical application of IL31^{K138A-SNAP}-IR700 both prevented and reversed Calcipotriol provoked dermatitis-like symptoms. This was evident as a return to baseline levels of scratching behavior (FIGS. 3i and k) and a normalization of skin structure and histology (FIGS. 3j and l). Thus molecule guided delivery of a photosensitizer complex allows for on-demand, pain-free control of chronic itch.

Finally, photo-ablation guided by the mutated IL31 according to the invention is specific for IL31RA expressing cells and does not affect other cell types such as keratinocytes or epidermal Langerhans cells (FIGS. 3q and r).

### Materials and Methods:

### Animals

Wild type or IL31RA knock out (IL31RA^{-/-}) Black 6/J, 8-10 week-old male mice were used for all behavioral studies. 1-3 day-old mice were used for primary keratinocyte culture. All mice were bred and maintained at the EMBL Mouse Biology Unit, Monterotondo, in accordance with Italian legislation (Art. 9, 27. Jan. 1992, no 116) under license from the Italian Ministry of Health, and in compliance with the ARRIVE guidelines.3

Production of Recombinant IL31^{SNAP} and IL31^{K138A-SNAP cDNAs for murine IL}31 and SNAP tag were cloned into pETM11 vector and expressed in E. Coli as fusion protein. To generate the mutant IL31^{K138A-SNAP} mutagenesis was performed, according to the manufacturer's instruction (Agilent, #200555). The proteins were isolated from inclusion bodies, solubilized, refolded, and eluted using a Ni-NTA resin (Qiagen, #30210). Eluted fractions were then pooled, concentrated and stored for further analysis.

### Synthesis of BG-IR700

3 mg of IRDye®700DX N-hydroxysuccinimide ester fluorophore (LI-COR Biosciences GmbH, Bad Homburg, Germany) were dissolved in 150 ul DMSO and treated with 1.5 mg BG-PEG11-NH₂ and 5 ul diisopropylethylamine. After 1 h, the product BG-PEG11-IRDye®700DX was purified by HPLC using a Waters Sunfire Prep C18 OBD 5 μM; 19×150 mm column using 0.1M triethylammonium acetate (TEAA)] (pH 7.0) and 0.1M TEAA in water/acetonitrile 3:7 (pH 7.0) as mobile phases A and B, respectively. A linear gradient from 100% A to 100% B within 30 minutes was used. The fractions containing the product were lyophilized.

### Primary Keratinocyte Culture

Primary keratinocytes were isolated from 1-3 day-old wild type and IL31RA^{-/-} mice as previously described (25). Briefly, newborn mouse skin was removed and incubated flat in cold and freshly thawed trypsin overnight, at 4° C., with the dermis side down. The next day, epidermis was peeled off, triturated and keratinocytes were cultured in serum free media (Invitrogen #10744-019) on Collagen I (Sigma #3867)-coated dishes (Ibidi #81151). All experiments were performed on 48-72 hours cultured cells.

### In-Vitro and In Vivo Labeling

For keratinocyte labelling, 1 μM IL31^{SNAP} or IL31^{K138A-SNAP} was coupled with 3 μM BG549surf (NEB #S9112) for 1 hour at 37° C. in CIB buffer (NaCl 140 mM; KCl 4 mM; CaCl₂ 2 mM; MgCl₂ 1 mM; NaOH 4.55 mM; Glucose 5 mM; HEPES to mM; pH 7.4). Cells were incubated with the coupling reaction for to minutes at 37° C., then washed 3 times in CIB; and imaged using confocal microscope.

For skin labelling, intradermal injection of 5 μM IL31^{SNAP} coupled with 5 μM BG549surf was performed in the nape of the neck. After to min (30 min) skin was collected, fix in PFA 4% overnight, OCT-embedded and cryo-sectioned (25 μm).

### In-Vitro Photo-Ablation

1 μM IL31^{SNAP} and 3 μM BG-IR700 were coupled for 1 hour at 37° C. The coupling reaction was applied for 10 minutes at 37° C. on primary wild type and IL31RA^{-/-} keratinocytes. Cells were then exposed to near infra-red light (680 nm) at 40 J/cm² for 2 minutes. 24 hours after light exposure cell death was assessed by Propidium iodide (PI) staining (Invitrogen #P3566) and cells were imaged with an epifluorescent microscope.

In-Vivo Photo-Ablation The skin at the nape of the neck of wildtype and IL31RA^{-/-} mice was shaved and injected with 5 μM IL31^{SNAP} or IL31^{K138A-SNAP} coupled to 15 μM BG-IR700 in a 50 μl volume. 20 minutes after the injection, near infra-red light (680 nm) at 120-150 J/cm² or at 550-600 J/cm² was applied at the injection site for 4 minutes. This procedure was repeated for 3 consecutively days. For other sensory modalities, the photoablation procedure was performed in the hind paw using a 20 μl injection volume. For histological analysis, mice were sacrificed after 3 days after the last illumination; skin was collected, fixed in PFA 4% and paraffin-embedded. 6 μm sections were stained for the TUNEL assay, following the manufacturer's instructions (Roche, #156792910).

### Microemulsion

The microemulsion was prepared as already described (24). Briefly, all the components were assembled as follow: Caprylic Triglyceride 81gr; Glyceryl Monocaprylate 27gr; Polysorbate80 12gr; Sorbitan Monooleate 8gr. The microemulsion was mixed with the coupling reaction (IL31^{K138A-SNAP}+IR700) at 1:1 ratio in 10 μl volume with 5 μM as IL31^{K138A-SNAP} final concentration.

### SDS-Page and Western Blot

To assess the coupling reaction, 10 and 20 pmol IL31^{SNAP} was coupled with 30 and 60 pmol BG549 respectively for 1 hour at 37° C. The coupling reactions were then loaded into a precast acrylamide gel (BioRad #456-9034), along with the same concentrations of IL31^{SNAP} alone. The bands corresponding to the binding of IL31^{SNAP} with BG549 were visualized by gel fluorescence. All the samples were visualized by Coomassie staining. Back skin from wild-type mice were injected with vehicle (PBS), 5 μM IL31^{SNAP} or IL31^{K138A-SNAP}. After 1 hour mice were sacrificed and skin was collected and lysated in Ripa Buffer (Sigma, #R0278) with proteases inhibitor cocktail. Protein lysate was quantified by Bradford assay. 30 μg total lysate were separated on 10% SDS-Page gel and transferred to a nitrocellulose membranes (Protran #10600007). Membrane were incubated with the following antibodies, anti STAT3 (Cell Signaling #9139), anti phospho STAT3 (Tyr705) (Cell Signaling #9131), anti MAPK (Cell Signaling #4695), anti phospho MAPK (Thr202/Tyr204) (Cell Signaling #9106), anti AKT (Cell Signaling #4691), anti phospho AKT (Ser473) (Cell Signaling #9271). Bands were visualized using the ECL detection system (Amersham #RPN2106); band density was calculated using ImageJ and the levels of phosphorylated proteins were normalized to the total counterpart.

### Atopic Dermatitis Model

10 μM analogue of the vitamin D3 analogue Calcipotriol (Tocris #2700) was topically applied on the shaved back skin of the mice for 10, 14 or 21 days, depending on the experiment. 0.002% DMSO was applied as vehicle control for the same duration of time. Atopic dermatitis development was assessed by histological analysis of the treated skin by Hematoxylin & Eosin staining, skin thickness measurement using a Caliper and scratching behavior over a 30 minute-recording period.

### Scratching Behavior

To evaluate the scratching response, 8-10 week-old male wild-type or IL31RA^{-/-} mice were shaved at the nape of the neck, placed in Plexiglas chambers to acclimatize (30 minutes), videotaped for 30 minutes and scratching bouts were counted. One bout was defined as an event of scratching lasting from when the animal lifted the hind paw to scratch until it returned it to the floor or started licking it. For every experiment, spontaneous scratching referred to as baseline, was measured. To characterize the mutant ligand in terms of itching properties, 5 μM IL31^{SNAP} or IL31^{K138A-SNAP} were injected for 3 consecutively days and scratching bouts were counted every day. To assess the photoablation effect on the scratching response, 5 μM IL31 (Peprotech, #210-31), 10 mM histamine (Sigma, #H7250), 1 mM LY344864 (Abcam, #ab120592) or 12.5 mM Chloroquine (Sigma, #C6628) were injected into the back skin of the mice previously injected with 5 μM ^{IL}31^{K138A-SNAP}+15 μM IR700 with or without near IR illumination. The injection of pruritogens was done 3 days after the last illumination treatment. For long-term reversal IL31 evoked scratching, IL31 was injected 1 day after the last day of illumination and then every week for 8 weeks. 10 μM Calcipotriol-mediated itch was also considered at different time point to monitor dermatitis development, with or without photoablation.

### Von Frey Test

Mice were habituated on an elevated platform with a mesh floor for 30 minutes. The plantar side of the hind paw was stimulated with calibrated von-Frey filaments (North coast medical #NC12775-99) to assess baseline levels of mechanical sensitivity. The stimulation was then repeated at the 3^{rd} day after the last photoablation performed on the same paw considered for the baseline. As a control group, the animals were injected but not illuminated. The 50% paw withdrawal thresholds were calculated using the Up-Down method (26).

### Hot Plate Test

Mice were injected for three consecutive days with IL31^{K134-SNAP} coupled with BG-IR700 with or without near IR light illumination. 3 days after the last injection, mice were placed on top of a hot plate (Ugo Basile #35150) that was preset to 52° C. and the latency to response as distinguished by flicking or licking of the hind paw was observed. In order to avoid injury to the mice, a cutoff of 30 seconds was set.

### Statistical Analysis

All statistical data are presented as Standard error of the mean (SEM) along with the number of samples analysed (n). Student's t-test and/or analysis of variance ANOVA were used; Statistical significance was assumed at p<0.05.

### II: PAIN

### Example 5: TrkB Positive Neurons are a Subset of Mechanoreceptive Neurons

Using TrkBCreERT2::Rosa26RFP reporter mice the inventors examined colocalization of TrkB with established cellular markers in adult sensory ganglia. Approximately 10% of dorsal root ganglia (DRG) were positive for TrkBCreERT2, corresponding to the ˜8% of cells which expressed TrkB mRNA (FIG. 1I). Expression was evident in 2 populations of large neurons marked by NF200 and NF200 plus Ret (FIG. 4A, B, I), and not present in nociceptors positive for CGRP or IB4, or C low threshold mechanoreceptors marked by TH (FIG. 4C-E, I). The inventors further investigated the projections of TrkB neurons to the skin and spinal cord. TrkBCreERT2 fibres extended to Meissner corpuscles in the glabrous skin (FIG. 4F) and formed longitudinal lanceolate endings around hair follicles (FIG. 4G). To assay TrkBCreERT2 positive sensory input into the spinal cord a reporter line was generated in which Cre dependent expression of mCherry was driven from the sensory neuron specific Avil locus. TrkBCreERT2::AvilmCherry positive sensory neurons were present in laminae III/IV of the dorsal horn of the spinal cord where they formed column-like structures extending dorsally (FIG. 4H). The authors also examined expression of TrkB in human tissue using a TrkB antibody. In agreement with mouse data, TrkB immunoreactivity was present in human DRG in large neurons co-expressing NF200 and Ret but largely absent from nociceptors expressing TrkA (FIG. 4i-k, n). Similarly, in glabrous skin, TrkB immunoreactivity was detected in NF200 positive fibers innervating Meissner corpuscles (FIG. 4l). Collectively, these data indicate that TrkBCreERT2 marks a population of putative mechanoreceptive neurons in mouse and human.

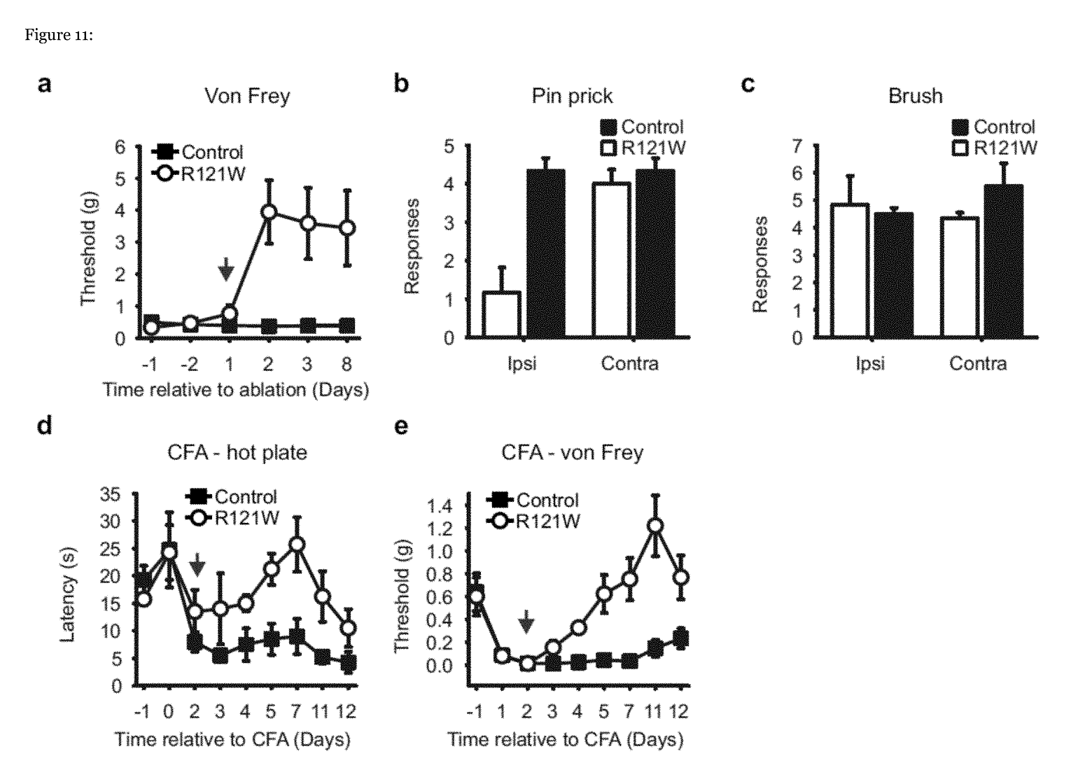

To unequivocally establish the identity of TrkBCreERT2 positive sensory neurons the inventors characterized their response properties utilizing a combination of electrophysiology and optogenetic activation. Mice expressing the light-gated ion channel channel-rhodopsin in TrkB positive cells were generated (TrkBCreERT2::Rosa26ChR2) and an ex vivo skin nerve preparation used to identify neuronal subtypes which could be concomitantly activated by light. Strikingly, the inventors determined that all D-hair and RAMs could be stimulated by light (FIG. 40-q) whereas all other subtypes of sensory neurons were not responsive (FIG. 40-q). Thus TrkB marks myelinated neurons that innervate hair follicles and are tuned to detect gentle moving mechanical stimuli.

### Example 6: Ablation of TrkB Positive Neurons Affects Exclusively Light Mechanical Sensation

To determine the role played by TrkBCreERT2 positive D-hairs and RAMs in sensory evoked behavior, the inventors genetically ablated TrkB neurons in the peripheral nervous system. A Cre-dependent diphtheria toxin receptor transgene knocked-in to the sensory neuron specific Avil locus was generated that allowed for selective deletion of TrkB positive neurons only in adult sensory ganglia. Upon systemic injection of diphtheria toxin a ˜90% ablation of TrkBCreERT2::AviliDTR and TrkB mRNA positive neurons was achieved with a parallel reduction in the number of NF200 positive neurons by ˜40% and no change in the expression of other markers (FIG. 5A-C).

The inventors performed a series of behavioral tests in these animals examining sensory responses to a range of thermal and mechanical stimuli. There was no difference in responses to evaporative cooling evoked by acetone application (FIG. 5D), or in thresholds to noxious heat (FIG. 5E) after diphtheria toxin ablation. Similarly, grip strength (FIG. 5F) was unaltered by ablation of TrkBCreERT2 neurons, as were responses to noxious pinprick (FIG. 5G), and static mechanical stimulation of the hairy skin evoked by application of tape to the back (FIG. 5H). Further examined were the responses to dynamic mechanical stimuli by monitoring responses to brushing of the plantar surface of a paw. Using a puffed out cotton swab which exerts forces in the range 0.7-1.6 mN, the inventors observed a significant reduction in responsiveness upon ablation of TrkB positive neurons (FIG. 5I). Intriguingly, these differences were not apparent upon application of stronger dynamic forces using a paint brush (>4 mN, FIG. 6B, D). Thus under basal conditions, TrkB positive sensory neurons are required for behavioral responses to the lightest of dynamic mechanical stimuli.

### Example 7: TrkB Sensory Neurons are Necessary and Sufficient to Convey Mechanical Pain in a Neuropathic Pain Model

On account of the exquisite sensitivity of TrkB positive neurons, the inventors next asked whether they contribute to mechanical hypersensitivity in models of injury induced pain. The inventors took both a loss of function approach using genetic ablation, and a gain of function approach using optogenetic activation of TrkB neurons. First considered was a model of inflammatory pain by injecting Complete Freund's Adjuvant (CFA) into the plantar surface of the paw, and monitoring responses to von Frey filaments and dynamic brush stimuli. Ablation of TrkB neurons in TrkBCreERT2::AviliDTR mice had no effect on either basal mechanical sensitivity or mechanical hypersensitivity after inflammation (FIG. 6A-B).

It was further examined whether optogenetic activation of TrkB neurons could evoke pain behavior under inflammatory conditions. Using stimulation parameters which evoked robust firing in the ex vivo skin nerve preparation, the inventors observed no discernible behavioral response to light application to the paw either in basal conditions or after inflammation in TrkBCreERT2::Rosa26ChR2 mice (FIG. 6C). Importantly, identical stimulation conditions applied to the auricle of the ear evoked a brief ear twitch in TrkBCreERT2::Rosa26ChR2 mice (not shown), likely reflecting activation of the dense network of mechanoreceptors in this structure.

Next neuropathic pain was induced in mice using the Spared Nerve Injury (SNI) model. Control mice developed a profound mechanical hypersensitivity in the sural nerve territory of the paw to both von Frey filaments and dynamic brush stimuli (FIGS. 6D and E). Strikingly, upon ablation of TrkBCreERT2::AviliDTR sensory neurons, mice did not develop mechanical allodynia to either punctate or brushing stimuli, and mechanical sensitivity remained at preinjury levels throughout the observation period. The inventors performed further experiments in TrkBCreERT2::Rosa26ChR2 mice to optogenetically activate these neurons. Three days after injury it was observed that selective stimulation of TrkB neurons with light evoked behavior indicative of pain. This was evident as a prolonged paw withdrawal from the stimulation, lifting of the paw and licking of the illuminated area (FIG. 6F) that continued for several minutes after light application. Such behavior persisted throughout the 2 weeks observation period and was never observed in control mice (FIG. 6F).

As a neuronal correlate of this apparent pain behavior, the inventors examined induction of the immediate early gene C-fos in the dorsal horn of the spinal cord. In TrkBCreERT2::Rosa26ChR2 mice without injury, optical stimulation evoked C-fos immunoreactivity primarily in laminae III and IV of the spinal cord, the region where TrkB neurons terminate (FIGS. 6G and I). Upon nerve injury however, identical stimulation parameters induced C-fos staining in lamina I of the dorsal horn, an area associated with nociceptive processing. Thus under neuropathic pain conditions, TrkB sensory neurons are necessary and sufficient to convey the light touch signal that evokes pain.

Example 8: Treatment of Mechanical Allodynia In Vivo

In light of the clinical importance of mechanical allodynia in neuropathic pain patients, it was sought to develop a pharmacological strategy to exploit the striking selectivity of TrkB to the peripheral neurons which provoke this pain state. It was reasoned that BDNF, the ligand for TrkB, may give access to these neurons and allow for their manipulation in wildtype, non-transgenic animals. To this end the inventors produced a recombinant BDNF protein with a SNAP-tag fused to its C-terminus that would enable its chemical derivatization. BDNF^{SNAP} was labelled in vitro with fluorescent SNAP-Surface647 substrate and applied to HEK293T cells expressing neurotrophin receptors. Fluorescently labelled BDNF^{SNAP} displayed remarkable selectivity for its cognate receptor complex TrkB/p75 (FIG. 7A), and did not bind to cells expressing related neurotrophin receptors TrkA/p75 (FIG. 7B) or TrkC/p75 (FIG. 7C).

The inventors further tested whether BDNF^{SNAP} would recognize native TrkB receptors in DRG neurons. BDNF^{SNAP} was conjugated to Qdot 655 quantum dots and applied to dissociated DRG from TrkBCreERT2::Rosa26RFP mice. a >95% overlap between BDNF^{SNAP} and TrkBCreERT2 positive cells (FIG. 7D) was observed indicating that recombinant BDNF^{SNAP} is a highly selective means of targeting TrkB neurons.

To manipulate TrkB neurons in vivo, it was reasoned that BDNF^{SNAP} may allow for targeted photoablation of these neurons through delivery of a photosensitizing agent. The inventors synthesized a benzylguanine modified derivative of the highly potent near-infrared photosensitizer IRDye®700 DX phthalocyanine (IR700) and conjugated it in vitro to BDNF^{SNAP}. In initial experiments BDNF^{SNAP-IR700} was applied to HEK293T cells expressing TrkB/p75 and cell death assayed following near infrared illumination. In cells expressing TrkB/p75 the inventors observed substantial cell death 24 hours after brief illumination (FIG. 7E) that was not evident upon mock transfection or treatment with IR700 alone (FIG. 7F).

The inventors next sought to assess the therapeutic potential of this approach by investigating the effects of BDNF^{SNAP}-IR700 mediated photoablation in wildtype mice with neuropathic pain. Upon establishment of robust mechanical allodynia three days after SNI, a range of concentrations of BDNF^{SNAP}-IR700 was injected into the ipsilateral paw of injured mice and the skin illuminated with different light intensities. Strikingly, the inventors observed a concentration and illumination dependent rescue of both von Frey withdrawal thresholds (FIG. 8a) and dynamic brush or cotton swab evoked allodynia (FIG. 8b and c) that persisted for more than 3 weeks after a single treatment regime. It was examined whether such pronounced effects were also evident in other types of neuropathic pain. Indeed, in both the streptozotocin model of painful diabetic neuropathy, and the paclitaxel model of chemotherapy induced neuropathic pain, a marked reversal of mechanical hypersensitivity that peaked around 10 days post treatment and returned to injury levels by day 20 (FIG. 8d and e) was observed. To determine the selectivity of this approach, the inventors further assessed the effects of BDNF^{SNAP}-IR700 mediated photoablation on behavioral responses under basal conditions. No deficits in sensitivity to cold, heat, or pinprick upon treatment were observed (FIG. 8f to h). Responses to cotton swab were also unaffected by photoablation (FIG. 3i), perhaps because the skin area that is stimulated in this test extends beyond the zone of illumination.

To investigate the mechanism by which BDNF^{SNAP}-IR700 reverses mechanical allodynia, a TrkB^{CreERT2}::Ros26^{SNAPCaaX} reporter mouse line was used to identify TrkB positive afferents, and a PGP9.5 antibody to label all fibers, in order to examine the innervation density of hypersensitive skin over the course of phototherapy. Prior to photoablation, TrkB positive lanceolate endings were detected around hair follicles (FIG. 9a) and innervating Meissner corpuscles in the plantar surface of the paw. At 7 days after photoablation (13 days post-SNI) when behavioral reversal of mechanical hypersensitivity was most pronounced, a selective loss of TrkB fibers but persistent innervation by PGP9.5 fibers in hairy and glabrous skin was observed (FIG. 9b, f, g). Indeed, many hair follicles displayed a complete loss of TrkB innervation but still contained PGP9.5 positive circumferential and longitudinal lanceolate endings demonstrating the remarkable specificity of ablation (FIG. 9c). At 24 days post-photoablation when mechanical hypersensitivity had reverted, TrkB positive fibers were again seen innervating their appropriate end organs in both glabrous and hairy skin (FIG. 9d). Importantly, there was no apparent reduction in innervation of control tissue injected with unconjugated IR700 and illuminated (FIG. 9f). It was further investigated whether loss of TrkB^{CreERT2} neurons was also evident at the level of the cell soma by analyzing the number of TrkB^{CreERT2} positive neurons in the DRG. No differences in the proportion of TrkB neurons 10 days after photoablation were observed (FIG. 9e and f), indicating that the loss of fibers likely reflects local retraction from their peripheral targets.

TrkB is also expressed by other cells in the skin in addition to sensory fibers. The inventors sought to identify these cell types and determine whether they are lost upon photoablation and contribute to the behavioral phenotype. TrkB was not detected in Merkel cells, keratinocytes, or dendritic and dermal antigen presenting cells, and BDNF^{SNAP}-IR700 mediated photoablation did not alter their numbers in the skin (FIG. 9g). Expression of TrkB was however evident in cells labelled with CD34, a marker of mast cells and epithelial and endothelial progenitor cells. Moreover, photoablation significantly reduced the number of CD34 positive cells in the skin (FIG. 9g). To determine whether it is loss of these cells or TrkB+ afferents which influences sensory behavior, BDNF^{SNAP}-IR700 was injected into the sciatic nerve at mid-thigh level and the nerve illuminated to ablate TrkB sensory fibers but spare CD34 cells in the skin. In these animals, behavioral responses to cooling, heating and pinprick were normal (FIG. 9h-j), however, sensitivity to cotton swab was reduced (FIG. 9k), paralleling the results using genetic ablation. It was further investigated whether optogenetically evoked pain behavior in SNI mice is dependent upon CD34 + cells or TrkB+ fibers in the skin. Upon photoablation of TrkB+ fibers in the sciatic nerve a significant reduction in light driven nocifensive behavior in TrkB^{CreERT2}::Rosa26^{ChR2} mice (FIG. 9l) was observed. Thus, TrkB+ sensory afferents, rather than other cells in the skin likely underlie behavioral sensitivity to light touch under basal conditions and after nerve lesion.

In summary, the results identify the first relay station in the neuronal pathway that confers pain from gentle touch under neuropathic pain states. It was demonstrated that TrkBCreERT2 positive sensory neurons detect the lightest touch under basal conditions but after nerve injury are both necessary and sufficient to drive mechanical allodynia.

The invention further describes a new technology based upon ligand mediated delivery of a phototoxic agent to target these neurons and reverse mechanical hypersensitivity in neuropathic pain states. This approach is analogous to clinically approved capsaicin patches, in which a high concentration of capsaicin is applied to the skin and leads to retraction of nociceptive fibers. Instead, here the invention targets directly the neurons responsible for mechanical allodynia, allowing for local, on demand treatment of pain through application of light.

### Example 9: Treatment of Inflammatory Pain using NGF to Target Nociceptive Sensory Neurons

A similar experiment as with BDNF was performed with nerve growth factor (NGF). The reasoning here was that TrkA, the receptor for NGF is expressed exclusively by nociceptive sensory neurons, thus their ablation should allow for treatment of acute and inflammatory pain. In initial experiments, a recombinant NGF^{SNAP} protein was produced and shown to bind to TrkA positive cells and not TrkB or TrkC. NGF^{SNAP} was conjugated to IR700 and injected into the paw of mice which was then illuminated with near IR light in the treatment group, while a control group received no illumination. Complete Freund's adjuvant (CFA) was injected into the paw, and responses to thermal stimuli monitored for a period of 50 days. In the control group NGF itself produced a robust thermal hyperalgesia which was further increased by CFA injections. In animals illuminated with near IR light, thermal hyperalgesia did not develop (FIG. 10a). In a further experiment, CFA was injected first and then the paw was subjected to NGF^{SNAP}-IR700 mediated photoablation and mechanical hypersensitivity was monitored. In control animals which received no illumination, robust mechanical hypersensitivity developed which was maintained throughout the 25 day observation period. In animals which received near IR light, and substantial reduction in mechanical withdrawal thresholds was observed and mechanical sensitivity returned to baseline levels (FIG. 10b).

A conceptual problem with using NGF^{SNAP} as a means of targeting TrkA positive nociceptors is that it in itself evokes pain and sensitization. To circumvent this problem, the authors generated an engineered NGF^{SNAP} with Arginine mutated to Tryptophan at position 121 (NGF^{R121W-SNAP}). This molecule was found to bind specifically to Hek293 cells expressing TrkA and p75 receptors (FIG. 10c) and to evoke cell death when conjugated to IR700 and applied to these cells and illuminated (FIG. 10d). Importantly, when NGF^{R121W-SNAP} was injected into the paw of mice it did not provoke mechanical hypersensitivity, while wildtype NGF^{SNAP} had a strong sensitizing effect (FIG. 10e). Thus NGF^{R121W-SNAP} is a “painless” NGF derivative that binds to TrkA receptors but does not activate pain signaling pathways.

To determine whether NGF^{R121W-SNAP} can be used as a photosensitizing agent to control pain, the authors conjugated it to IR700 and injected it into skin for subsequent near IR light illumination. Acute behavioral responses were first examined. NGF^{R121W-SNAP} mediated photoablation was found to significantly elevate painful mechanical withdrawal latencies to von Frey filaments, while non-illuminated controls showed no change (FIG. 11a). Similarly, responses to painful pinprick were reduced by photoablation (FIG. 11b) while non-nociceptive responses to brush were not affected (FIG. 11c). Finally, the authors examined the efficacy of photoablation under inflammatory pain conditions. Using the CFA model of inflammatory pain, it was found the NGF^{R121W-SNAP}-IR700 mediated photoablation led to prolonged recovery of both thermal hyperalgesia (FIG. 11d) and mechanical hypersensitivity (FIG. 11e) in this model. Thus NGF^{R121W-SNAP} can be used to control acute nociceptive pain and hypersensitivity that results from an inflammatory stimulus.

1. A conjugate compound for use in therapy for targeting and inhibiting/killing a target cell in a target body surface area of a subject, wherein the conjugate compound, preferably a protein conjugate compound, comprises (i) a binding domain which specifically binds to the target cell, and (ii) a photosensitive inhibition/cytotoxin group, and wherein the therapy comprises the steps of:
(a) Administering said conjugate compound to the subject, and
(b) Irradiating said target body surface area of the subject with an appropriate excitation light in an amount to effectively activate said photosensitive inhibition/cytotoxin group and to induce cellular inhibition or cell death of the target cell.

2. The conjugate compound for use according to claim 1, wherein the target cell is a neuron, preferably a sensory neuron.

3. The conjugate compound for use according to claim 1, wherein the binding domain specifically binds to a receptor expressed on the target cell.

4. The conjugate compound for use according to claim 3, wherein the binding domain is a receptor ligand, or a receptor binding fragment thereof, or a receptor binding antibody, or a receptor binding fragment thereof.

5. The conjugate compound for use according to claim 1, wherein the photosensitive cytotoxin group is a phthalocyanine dye IRDye® 700DX, or a derivative thereof, such as a benzylguanine modified derivative.

6. The conjugate compound for use according to claim 1, wherein the therapy is for alleviating a neurological sensation in the target body surface area of the subject, for example a neurological sensation selected from noxious or innocuous stimuli, such as all forms of mechanical (touch) sensation, pain and/or itching.

7. The conjugate compound for use according to claim 1, wherein conjugate compound comprises a pruritogen as a binding domain which specifically binds to the cell, for example interleukin-31 (IL31) or mutant IL31, or derivatives or fragments of these compounds.

8. The conjugate compound for use according to claim 7, wherein the mutant IL-31, is an IL31 binding to IL31 receptor (I131RA and OSMR), but eliciting a reduced IL31 signaling, such as a mutation in human IL31 at position K134, for example IL31^{K134A}.

9. The conjugate compound for use according to claim 1, wherein the binding domain which specifically binds to the target cell is capable to bind to an expression product of a TrkB gene, preferably the NTRK2 gene, in the target cell.

10. The conjugate compound for use according claim 9, wherein the TrkB ligand is a protein binding to the TrkB/p75 receptor complex, and preferably is selected from Brain-derived neurotrophic factor (BDNF) or Neurotrophin 4 (NT-4).

11. The conjugate compound for use according to claim 1, wherein the binding domain which specifically binds to the target cell is capable to bind to an expression product of a TrkA gene in the target cell.

12. The conjugate compound for use according to claim 11, wherein the compound that is capable to bind to an expression product of the TrkA gene comprises a TrkA-ligand or an anti-TrkA-antibody or anti-TrkA-T cell receptor (TCR), or chimeric antigen receptor (CAR); or wherein the compound comprises a nucleic acid having a nucleic acid sequence that is complementary to, or can under stringent conditions hybridize to, an mRNA produced by the TrkA locus.

13. The conjugate compound for use according claim 12, wherein the TrkA ligand is a protein binding to the TrkA receptor, and preferably is Nerve Growth Factor (NGF), more preferably a mutant NGF, such as a mutant human NGF, for example an NGF mutated at position R121, such as NGF^{R121W}.

14. The conjugate compound for use according to claim 1, wherein the conjugate compound in step (a) is administered locally or systemically to the subject, such as a subcutaneous injection, and intraneural injection, or topical administration, such as by applying a cream, ointment, salve, or other topical formulations.

15. A conjugate compound comprising (i) a binding domain which specifically binds to the target cell, and (ii) a photosensitive inhibition/cytotoxin group , wherein the binding domain is selected from a TrkA ligand, a TrkB ligand, or a pruritogen.

16. A pharmaceutical composition comprising a conjugate compound according to claim 15, together with a pharmaceutically acceptable carrier and/or excipient.
